SEQUENCE LISTING (42 sequences)

SEQ ID NO: 1 - protein, 189 aa, Streptomyces sp. KNK269
  MISC_FEATURE: amino acid sequence of subunit gamma of aldehyde oxidase
GADLEPEPVVDEHSTVTLNVNGDPTTLTVDHRTTVLETLRESFGLTGAKKGCDHGQCGACTVLVDGRRVNSCLLLAVAQDGATITTVEGLADGEDLHPVQRAFVERDALQCGFCTPGQICSAVGMLREAADGHPSHVTGPDTAAGPDAATGPGGPVALDADEIRERMSGNIFRCGAYHRIVEAIEDVIE

SEQ ID NO: 2 - protein, 333 aa, Streptomyces sp. KNK269
  MISC_FEATURE: amino acid sequence of subunit beta of aldehyde oxidase
MKPFGYVRPASPDEAVRLCAEGSGARFLGGGTNLVDLMKLGVEAPRVLIDVAGLPLDRVTRTADGGLAIGATVRNSDLAAHPDVTDRYPVLSQALLAGASGQLRNSATTGGNLLQRTRCRYFQDVSKPCNKRLPGSGCPARDGTHRDLAILGHSPECVATNPSDMAVALAALDATVVLLGPEGERAVPLTEFHRLPGENPDQDTAIRTGELITEVVLPPPAPGAATRYRKARDRASFAFALVSVAATLRVDAGRVEHATLAFGGVAHRPWRARRAEAELRGAPATPAVFQHALIAELAEARPLRDNVFKVDLARRLALDVLGELTERQPTRTG

SEQ ID NO: 3 - protein, 693 aa, Streptomyces sp. KNK269
  MISC_FEATURE: amino acid sequence of subunit alpha of aldehyde oxidase
MTTRAPQFPGAPVVRREARDKVTGTARYAADRTPAGCLYAWTVPAAVAAGRVTAVRAVDALAVPGVHTVLTHDNAPELREPDDPILAVLQDDRVPHHGWPIALVIAESPEAARTGAGELHVEYARDEHDVTLTEDHPGLYTPEEANGGEPAVRVRGDVERALAASPVQVDATYRMVALHNHPMEPHAATAVRADGHLTVHDSSQGSTKVRESLAQAFGLAADRITVVSEHVGGGFGSKGTTRPQGVLAAMAAQHTGRPVKLVFPRAQLAEVVGHRAPTIQRVRLGADLDGVLTAVSHEVVTQTSTVKEFVEQAAVPARVMYASPHSRTAHRVTALDVPSPSWMRAPGEASGMYALESAMDVLDSALGLDPVELRLRNEPETEPDSGRPFSSRGLAACLRDGAARFGWQDRDPRPAARQEGRWLIGTGVASATYPVLLSRSTASAHATRDGDLSIAVNATDIGTGARTVLAQIAADVLGVAPEALRVDIGSTDLPAAPLAGGSSGTASWGWSVHKAATALAARLAEHRGPLPADGITTTADTGQETGEESPYARHAFGAHFAETAVDAVTGEVRVRRLLGVYAAGRILNERTARSQFTGGMVMGIGMALTEGCGIDPGFGDFTAKDLASYHVPVCADTADIQAHWIEEDDRHLNPMGSKGIGEIGIVGTAAAIGNAVRHATGARLRELPLTTDT

SEQ ID NO: 4 - DNA, 537 bp, Streptomyces sp. KNK269
  misc_feature: DNA sequence of subunit gamma of aldehyde oxidase
gaacactccaccgtgaccctgaacgtcaacggcgacccgacgacgctgaccgtcgaccaccgcaccacggtcctggagacgctgcgcgagagcttcgggctcaccggggccaagaagggctgcgaccacggccagtgcggggcctgcacggtcctcgtcgacgggcggcgggtcaacagctgcctgctgctcgccgtcgcccaggacggcgccacgatcaccaccgtggaagggctcgccgacggcgaggacctgcacccggtgcagcgggcgttcgtcgagcgcgacgccctccagtgcggcttctgcaccccgggccagatctgctcggccgtgggcatgctccgcgaggccgcggacggccacccctcgcacgtcaccggaccggacaccgccgccggaccggacgccgctaccggaccgggcgggcccgtcgcactcgacgcggacgagatccgggaacggatgagcggcaacatcttccggtgcggggcctatcatcggatcgtagaagcgatcgaggacgtgatcgagtga

SEQ ID NO: 5 - DNA, 1002 bp, Streptomyces sp. KNK269
  misc_feature: DNA sequence of subunit beta of aldehyde oxidase
gtgaaaccgttcggctatgtgcgccccgcctcccccgacgaggctgtccggctctgcgcggaaggctccggcgcccgcttcctgggcggcggcaccaatctggtggacctgatgaagctcggcgtcgaggcgccccgggtcctcatcgacgtcgcagggcttccgctggaccgggtgacccggacggccgacggcggcctcgccatcggcgccaccgtgcgcaacagcgacctcgccgcccaccccgacgtgacggaccggtaccccgtgctgtcccaggcgctgctggccggggcctccggtcagctccgcaactccgccaccaccggcggcaacctgctccagcgcacccggtgccgctacttccaggacgtctccaagccctgcaacaaacggcttcccggctccggctgccccgcacgcgacggcactcaccgcgacctcgcgatcctcgggcactcgccggagtgcgtggccaccaacccgtccgacatggccgtggccctcgccgcgctcgacgcgaccgtggtgctgctggggcccgagggcgaacgggccgtcccgctcaccgagttccaccggttgccgggggagaaccccgaccaggacaccgcgatcaggaccggcgagctgatcaccgaggtggtgctgccgccgccggcgcccggggcggccacccgctaccgcaaggcccgcgaccgcgccagcttcgccttcgcgctcgtctccgtcgccgccacgctccgggtcgacgccggccgcgtcgagcacgccaccctcgcgttcggcggcgtcgcccaccggccgtggcgggcccgcagggccgaggccgagctgcgcggcgccccggccacgcccgccgtgttccagcacgccctgatcgccgaactggccgaggcgcgaccgctgcgcgacaacgtcttcaaggtggacctcgcccgccggctcgccctcgacgtgctcggcgaactcaccgagcggcaacccacccggaccggctga

SEQ ID NO: 6 - DNA, 2055 bp, Streptomyces sp. KNK269
  misc_feature: DNA sequence of subunit alpha of aldehyde oxidase
atgaccacccgagccccgcagttccccggtgcgcccgtcgtccgcagagaggcccgggacaaggtcaccggcaccgcccgctacgccgccgaccgcacgcccgccggctgcctctacgcctggaccgtcccggccgccgtcgccgccggacgcgtcaccgccgtacgggccgtcgacgctctcgccgtccccggcgtccacaccgtcctcacccacgacaacgcgcccgaactccgggagcccgacgacccgatcctcgccgtgctccaggacgaccgggtcccgcaccacggctggcccatcgccctcgtgatcgcggagtccccggaggccgcccggacaggggccggggagctccacgtcgaatacgcacgcgacgagcacgacgtgaccctcaccgaagaccatccgggtctctacacgcccgaggaggccaacggcggtgagcccgccgtccgggtgcgcggcgacgtggaacgggccctggcggcctcgcccgtccaggtcgacgccacttaccggatggtcgcgctgcacaaccaccccatggaaccgcacgccgccaccgcggtgcgggccgacgggcacctcaccgtccacgactccagccagggctccaccaaggtccgcgagagtctggcccaggcgttcggactggcggcggaccggatcaccgtggtctccgagcacgtcggcggcggcttcggctcgaagggcaccacccgcccccagggagtcctggcggcgatggccgcccagcacaccgggcggccggtgaagctggtgttcccccgcgcccagctggccgaggtcgtcggccaccgcgcccccaccatccagcgagtcaggctcggcgcggacctggacggggtgctcaccgcggtcagccacgaggtcgtcacccagacctccaccgtgaaggagttcgtcgagcaggccgccgtgcccgcccgcgtcatgtacgcctcgccgcacagccggaccgcgcaccgggtgaccgccctcgacgtccccagcccctcctggatgcgcgccccgggggaggcctccgggatgtacgccctggagtccgccatggacgtgctggactccgcactcggcctggaccccgtcgagctgcggctccgcaacgaaccggagaccgaaccggacagcggccgccccttcagcagccggggcctcgccgcctgtctgcgcgacggcgcggcccggttcggctggcaggaccgcgacccccggcccgcagcccgccaggaaggccgctggctcatcggtacgggcgtggcctcggcgacctaccccgtcctcctctcccggtccaccgcgagcgcccacgccacccgggacggtgacctgagcatcgccgtcaacgccaccgacatcggcaccggggcccgcaccgtcctggcccagatcgccgccgacgtgctcggcgtcgccccggaggccctgcgcgtcgacatcggctccaccgacctgcccgccgccccgctggccggaggctcctccggcaccgcctcctggggctggtccgtgcacaaggcggcgaccgccctggccgcccgcctcgcggagcaccggggccccctgcccgccgacgggatcaccaccaccgccgacaccggacaggagaccggcgaggagtccccgtacgcccggcacgccttcggcgcgcacttcgccgagacggccgtcgacgccgtcacgggcgaggtgcgcgtacgccgcctcctcggcgtgtacgccgccgggcggatcctcaacgagcgcaccgcacgctcccagttcaccggtggcatggtgatgggcatcggcatggccctcaccgagggctgcggcatcgacccgggcttcggcgacttcaccgccaaggacctcgcctcctaccacgtgcccgtctgcgccgacacggccgacatccaggcgcactggatcgaggaggacgaccgccacctcaacccgatgggcagcaagggcatcggcgagatcgggatcgtcggcaccgccgccgccattggcaacgcggtccgccacgccaccggcgcccggctgcgc

SEQ ID NO: 7 - protein, 1115 aa, Microbacterium sp. KNK011
  MISC_FEATURE: amino acid sequence of aldehyde oxidase containing signal peptide
MPVPAVLSGPRRRSTRVARTLAAGLLGGALVATGAILPAGAAVAPAVNTPADLPKQEPGVTLRTYSTPPLTELCTLKSGQTPNVDKLMSTIDWRTDEQFGAGDNFITHALANLTVSTPGQYAFRLTSDDGSRLTLDGTQLIDNDGLHGAESVEGTVTLDVGVHDLFVEMFEATNGQQLTVEWKVPGSSSFTVIPNSVLSTEAGVVRVTAPGTKYCEGSTDSAGDGLRLDAVNPNYTLVNLRPEGFTPKVSGLAFLGNGDLAVLTTGSVNSGGWDTSTPGKVFVLKGAQAADGPEDVTVVEAAGGLLNPMGIDVIDDKIYVSERYQLTELSDTNGDGSYETKRKVAEYPSGNNFHEFAFGLIHDEKNFYVNLSVAIDNGGATTNPQPAKNRGTSVKIDRATGAISYVAGGLRTPNGVAFGPENELFAMDNQGAWLPANKLVNVKQDRFFNHYTNPAGPFDQNPVTAPVVWIPQNEIGNSPSTPIMLKDGPFAGQMMFGDVTYGGLQRAFLEKVDGEFQGAVFRHSAGFEVGVNRVIEGPDTSLYIGGTGEGGNWGEAGKLTYGLQKLVPNTTQNAFDMKSMKVVEGGFEIEYTQPLSDETLANLASAYRAEQWRYLPTSTYGGPKVDEEILSLTGATASADRKTVTVSFDGLKQGRVVHLRSPQPFAAASGDTLWSTEAWYTLNSLPGYVSPADQGWYEAEEARLIGGAKFDAEHSGYSGAGFAGGMWQAGSAFEFTVNAETAGTVPVSVRYSNGPNPAPGSKDVNLYVNGQNLGKWDFASTGDWKTWATITRDMPLVAGTNTIALKYETGNKGNINVDVLSIGTADICAPSQVEDGYRPLFDGTLESLNAGWRMAGPGGFGRQNDCSIRGEGGMGLLWHKAQELNEYSLKLDWKLIADHNGGVFVGFPDPKNDPWIAVNQGYEIQIDASDAADRTTGAIYTFQGADADAVKASLKPVGQWNAYEIVVKGQTIKIFLNGTLVNDFTSTDPARDLSQGFIGLQNHGGGEAVSYRNVRVKDIDEPAPLAVTASAEVKCLAKKASVTARATNTDTVPVDVTLTTAWGERVIPAVQPGATVFHTFTTRAASVPAGEATVTATGDGRTGGATASYAAKSCG

SEQ ID NO: 8 - protein, 1069 aa, Microbacterium sp. KNK011
  MISC_FEATURE: amino acid sequence of aldehyde oxidase
VNTPADLPKQEPGVTLRTYSTPPLTELCTLKSGQTPNVDKLMSTIDWRTDEQFGAGDNFITHALANLTVSTPGQYAFRLTSDDGSRLTLDGTQLIDNDGLHGAESVEGTVTLDVGVHDLFVEMFEATNGQQLTVEWKVPGSSSFTVIPNSVLSTEAGVVRVTAPGTKYCEGSTDSAGDGLRLDAVNPNYTLVNLRPEGFTPKVSGLAFLGNGDLAVLTTGSVNSGGWDTSTPGKVFVLKGAQAADGPEDVTVVEAAGGLLNPMGIDVIDDKIYVSERYQLTELSDTNGDGSYETKRKVAEYPSGNNFHEFAFGLIHDEKNFYVNLSVAIDNGGATTNPQPAKNRGTSVKIDRATGAISYVAGGLRTPNGVAFGPENELFAMDNQGAWLPANKLVNVKQDRFFNHYTNPAGPFDQNPVTAPVVWIPQNEIGNSPSTPIMLKDGPFAGQMMFGDVTYGGLQRAFLEKVDGEFQGAVFRHSAGFEVGVNRVIEGPDTSLYIGGTGEGGNWGEAGKLTYGLQKLVPNTTQNAFDMKSMKVVEGGFEIEYTQPLSDETLANLASAYRAEQWRYLPTSTYGGPKVDEEILSLTGATASADRKTVTVSFDGLKQGRVVHLRSPQPFAAASGDTLWSTEAWYTLNSLPGYVSPADQGWYEAEEARLIGGAKFDAEHSGYSGAGFAGGMWQAGSAFEFTVNAETAGTVPVSVRYSNGPNPAPGSKDVNLYVNGQNLGKWDFASTGDWKTWATITRDMPLVAGTNTIALKYETGNKGNINVDVLSIGTADICAPSQVEDGYRPLFDGTLESLNAGWRMAGPGGFGRQNDCSIRGEGGMGLLWHKAQELNEYSLKLDWKLIADHNGGVFVGFPDPKNDPWIAVNQGYEIQIDASDAADRTTGAIYTFQGADADAVKASLKPVGQWNAYEIVVKGQTIKIFLNGTLVNDFTSTDPARDLSQGFIGLQNHGGGEAVSYRNVRVKDIDEPAPLAVTASAEVKCLAKKASVTARATNTDTVPVDVTLTTAWGERVIPAVQPGATVFHTFTTRAASVPAGEATVTATGDGRTGGATASYAAKSCG

SEQ ID NO: 9 - DNA, 3348 bp, Microbacterium sp. KNK011
  misc_feature: DNA sequence of aldehyde oxidase containing signal peptide
atgcccgtacccgctgtcttgtccggcccccgccggcgcagcacccgtgtcgcgaggaccctcgcggccggtctcctgggcggagcactcgtggccacaggagcgatcctgcccgccggtgccgccgtcgcccccgccgtcaacaccccggcggacctgcccaagcaagaaccgggtgtgacgcttcgcacctactccaccccgcccctcaccgagctctgcacgctgaagtcgggccagaccccgaacgtcgacaagctcatgtcgacgatcgactggcgcaccgacgagcagttcggcgcgggcgacaacttcatcacgcacgccctggccaacctcaccgtcagcacgcccggtcagtacgccttccgcctgacgagcgacgacggctcacgcctcaccctcgacggcacgcagctgatcgacaacgacggactgcacggagcggaatccgtcgagggcaccgtcacgctcgacgtgggagtccacgacctgttcgtcgagatgttcgaggccaccaacggccagcagctcaccgtcgaatggaaggtcccgggctcctcgagcttcaccgtcatccccaacagcgtgctcagcaccgaggcgggtgtcgtgcgcgtcaccgcccccgggacgaagtactgcgagggcagcaccgactccgccggcgacggtctgcgcctggatgccgtgaaccccaactacacgctcgtgaacctgcgccccgagggcttcacccccaaggtgtcgggcctggccttcctcggcaacggcgatctcgccgtgctgaccaccggctcggtcaactccgggggatgggacacctcgacccccggcaaggtcttcgtgctgaagggcgcccaggcggccgacggacccgaggacgtcaccgtcgttgaggcggccggcggtctgctgaacccgatgggcatcgacgtcatcgacgacaagatctacgtctcggagcgctaccagctcaccgagctcagcgacacgaacggcgacggctcgtacgagacgaagcgcaaggtcgccgagtacccctcgggcaacaacttccacgagttcgccttcggcctgatccacgacgagaagaacttctacgtcaacctctcggtcgcgatcgacaacggcggggcgaccaccaacccgcagccggcgaagaaccgtggcacctcggtcaagatcgaccgcgccaccggggcgatcagctacgtcgccggcggtctgcggacccccaacggcgtggccttcggtcccgagaacgaactcttcgcgatggacaaccagggtgcctggctgcccgcgaacaagctcgtcaacgtcaagcaggatcgcttcttcaaccactacacgaaccccgcgggcccgttcgatcagaaccccgtcacggctcccgtcgtgtggatcccgcagaacgagatcggcaactccccgagcacgcccatcatgctgaaggacggtcccttcgcgggacagatgatgttcggcgacgtcacctacggcggcctgcagcgcgccttcctcgagaaggtggacggtgagttccagggcgcggtcttccgccactccgcgggcttcgaggtcggcgtcaaccgcgtcatcgagggccccgacacgtcgctgtacatcggcggcaccggagaaggcggcaactggggtgaggcgggcaagctcacatacggcctgcagaagctcgtcccgaacaccacgcagaacgccttcgacatgaaatccatgaaggtcgtcgagggcggattcgagatcgagtacacccagcccctctccgacgagacgctggcgaacctcgcctcggcgtaccgggccgagcagtggcgctacctgcccacctccacctacggcggaccgaaggtcgacgaggagatcctctcgctcaccggcgcgacggcatccgccgaccgcaagacggtcacggtctcattcgacggtctgaagcaaggccgcgtcgtgcacctgcgctcgccgcagcccttcgccgcagccagtggcgacacgctgtggagcaccgaggcctggtacacgctcaactcgctgccgggctacgtctcgccggccgaccagggctggtacgaggccgaggaggcccgcctgatcggcggcgccaagttcgacgccgagcacagcggttactcgggagccggcttcgccggcggcatgtggcaggccggatccgcgttcgagttcacggtgaacgccgagaccgcgggcaccgtccccgtctcggtgcgctattccaacggcccgaaccccgcgcccggctccaaggacgtcaacctctacgtcaacggtcagaacctcgggaagtgggacttcgcctccacgggcgactggaagacctgggccacgatcacgcgggacatgccgctggtcgccgggacgaacaccatcgcgctgaagtacgagacgggcaacaagggcaacatcaacgtcgacgtcctgtcgatcggcaccgccgacatctgcgccccgtcgcaggtcgaggacggctaccgcccgctcttcgacggcacgctcgagagcctgaacgccgggtggcgcatggccggccccggcggcttcggtcgacagaacgactgcagcatccgcggcgaaggcggcatgggcctgctctggcacaaggcgcaggagctgaacgagtacagcctcaagctggactggaagctcatcgccgatcacaacggcggcgtcttcgtcgggttccccgacccgaagaacgacccgtggatcgcggtcaaccagggctacgagatccagatcgacgcgtccgacgccgccgatcgcaccaccggtgcgatctacaccttccagggtgcggatgccgacgcggtgaaggcctcgctcaagcccgtcggtcagtggaacgcgtacgagatcgtcgtgaaggggcagaccatcaagatcttcctcaacggcacgctggtgaacgacttcaccagcaccgatcccgctcgcgacctctcgcagggcttcatcggcctgcagaaccacggcggtggggaggcggtgtcgtaccgcaacgtccgcgtcaaggacatcgacgagccggcccccctcgcggtgaccgcctcggccgaggtcaagtgcctggccaagaaggcgtcggtgacggcccgcgcgaccaacaccgacacggtgccggtcgacgtgacgctcacgacggcgtggggcgagcgggtcatccccgccgtgcagcccggggcgacggtgttccacaccttcaccacccgcgccgcgtccgtccccgcgggcgaggcgacggtgaccgcgaccggcgacggtcgcaccggcggggccacggcctcgtacgcagccaagagctgcggctga

SEQ ID NO: 10 - DNA, 3210 bp, Microbacterium sp. KNK011
  misc_feature: DNA sequence of aldehyde oxidase
gtcaacaccccggcggacctgcccaagcaagaaccgggtgtgacgcttcgcacctactccaccccgcccctcaccgagctctgcacgctgaagtcgggccagaccccgaacgtcgacaagctcatgtcgacgatcgactggcgcaccgacgagcagttcggcgcgggcgacaacttcatcacgcacgccctggccaacctcaccgtcagcacgcccggtcagtacgccttccgcctgacgagcgacgacggctcacgcctcaccctcgacggcacgcagctgatcgacaacgacggactgcacggagcggaatccgtcgagggcaccgtcacgctcgacgtgggagtccacgacctgttcgtcgagatgttcgaggccaccaacggccagcagctcaccgtcgaatggaaggtcccgggctcctcgagcttcaccgtcatccccaacagcgtgctcagcaccgaggcgggtgtcgtgcgcgtcaccgcccccgggacgaagtactgcgagggcagcaccgactccgccggcgacggtctgcgcctggatgccgtgaaccccaactacacgctcgtgaacctgcgccccgagggcttcacccccaaggtgtcgggcctggccttcctcggcaacggcgatctcgccgtgctgaccaccggctcggtcaactccgggggatgggacacctcgacccccggcaaggtcttcgtgctgaagggcgcccaggcggccgacggacccgaggacgtcaccgtcgttgaggcggccggcggtctgctgaacccgatgggcatcgacgtcatcgacgacaagatctacgtctcggagcgctaccagctcaccgagctcagcgacacgaacggcgacggctcgtacgagacgaagcgcaaggtcgccgagtacccctcgggcaacaacttccacgagttcgccttcggcctgatccacgacgagaagaacttctacgtcaacctctcggtcgcgatcgacaacggcggggcgaccaccaacccgcagccggcgaagaaccgtggcacctcggtcaagatcgaccgcgccaccggggcgatcagctacgtcgccggcggtctgcggacccccaacggcgtggccttcggtcccgagaacgaactcttcgcgatggacaaccagggtgcctggctgcccgcgaacaagctcgtcaacgtcaagcaggatcgcttcttcaaccactacacgaaccccgcgggcccgttcgatcagaaccccgtcacggctcccgtcgtgtggatcccgcagaacgagatcggcaactccccgagcacgcccatcatgctgaaggacggtcccttcgcgggacagatgatgttcggcgacgtcacctacggcggcctgcagcgcgccttcctcgagaaggtggacggtgagttccagggcgcggtcttccgccactccgcgggcttcgaggtcggcgtcaaccgcgtcatcgagggccccgacacgtcgctgtacatcggcggcaccggagaaggcggcaactggggtgaggcgggcaagctcacatacggcctgcagaagctcgtcccgaacaccacgcagaacgccttcgacatgaaatccatgaaggtcgtcgagggcggattcgagatcgagtacacccagcccctctccgacgagacgctggcgaacctcgcctcggcgtaccgggccgagcagtggcgctacctgcccacctccacctacggcggaccgaaggtcgacgaggagatcctctcgctcaccggcgcgacggcatccgccgaccgcaagacggtcacggtctcattcgacggtctgaagcaaggccgcgtcgtgcacctgcgctcgccgcagcccttcgccgcagccagtggcgacacgctgtggagcaccgaggcctggtacacgctcaactcgctgccgggctacgtctcgccggccgaccagggctggtacgaggccgaggaggcccgcctgatcggcggcgccaagttcgacgccgagcacagcggttactcgggagccggcttcgccggcggcatgtggcaggccggatccgcgttcgagttcacggtgaacgccgagaccgcgggcaccgtccccgtctcggtgcgctattccaacggcccgaaccccgcgcccggctccaaggacgtcaacctctacgtcaacggtcagaacctcgggaagtgggacttcgcctccacgggcgactggaagacctgggccacgatcacgcgggacatgccgctggtcgccgggacgaacaccatcgcgctgaagtacgagacgggcaacaagggcaacatcaacgtcgacgtcctgtcgatcggcaccgccgacatctgcgccccgtcgcaggtcgaggacggctaccgcccgctcttcgacggcacgctcgagagcctgaacgccgggtggcgcatggccggccccggcggcttcggtcgacagaacgactgcagcatccgcggcgaaggcggcatgggcctgctctggcacaaggcgcaggagctgaacgagtacagcctcaagctggactggaagctcatcgccgatcacaacggcggcgtcttcgtcgggttccccgacccgaagaacgacccgtggatcgcggtcaaccagggctacgagatccagatcgacgcgtccgacgccgccgatcgcaccaccggtgcgatctacaccttccagggtgcggatgccgacgcggtgaaggcctcgctcaagcccgtcggtcagtggaacgcgtacgagatcgtcgtgaaggggcagaccatcaagatcttcctcaacggcacgctggtgaacgacttcaccagcaccgatcccgctcgcgacctctcgcagggcttcatcggcctgcagaaccacggcggtggggaggcggtgtcgtaccgcaacgtccgcgtcaaggacatcgacgagccggcccccctcgcggtgaccgcctcggccgaggtcaagtgcctggccaagaaggcgtcggtgacggcccgcgcgaccaacaccgacacggtgccggtcgacgtgacgctcacgacggcgtggggcgagcgggtcatccccgccgtgcagcccggggcgacggtgttccacaccttcaccacccgcgccgcgtccgtccccgcgggcgaggcgacggtgaccgcgaccggcgacggtcgcaccggcggggccacggcctcgtacgcagccaagagctgcggctga

SEQ ID NO: 11 - protein, 1107 aa, Cellulosimicrobium cellulans IFO 15516
  MISC_FEATURE: amino acid sequence of aldehyde oxidase containing signal peptide
MLERTSLPVSRRIRYRRGAAALTLGAVVVAGWAVPAAADLPEQEPGVTLRTFQLAQNPGAVCTLKSGQTPNVDKLMPTIDWSTAEQFGAEDNFISQVSANLHVPADGQYQFRVTNDDGALVYIDGQLVVENDGPNDSTSVEGSATLTAGVHDLRVDYYEGSDKQRLTLAWKTPGSSTFEVIPTSALSTEAGVVRVTAPGYKYCEGATDTAGDGLRLDSVNPNYDLVDLRPEGFEPKVSGLAFTPDEKLAVVTTGEVSSGGWRPDPVSGEVYFLDGVLDADGPEDVTATKVADELLNPMGIEVVEDSIFVSERYQLTQLTDPDGDGFYDTHTKIAEWPDGGNFHEFAFGLIHDEDYFYVNLSVAINNGGATTNPQPAANRGTSIKIDRETGEVTYVAGGLRTPNGIGFGPEDEIFATDNQGAWLPSNKLIHIQQDKFYNHYTNPAGPFDANPVTPPAVWLPQNEIANSPGNPILVEDGEFAGQMLLGDVTYGGIQRAFLEKVDGEFQGAVFRHTAGLEVGVNRVIYGPDGALYAGGTGEGGNWGESGKLRYGLQKLVPVNEDSFDMKEMRVVEGGFEIEYTDPVSDEVVEKLADAYQVKQWRYVPTQQYGGPKVDEEPLFVTDATVSEDRTTVTLTIDGLKPDHVVYIRSPRPFASAEGTELLSTEAWYTLNSLPGYVAPADRGWYEAELAQPLGSSSIGSDHSNYSGSGFAAGMTNVGAGRTFSVTVPEAGTYPVNVRYANGIHPYTSLRSKNVSLHVNGQDLGQWNFPTTGSWKDWGVQTRDLQLQAGTNTITLAYEAGDEGNINIDVLSIGENPDICSPGEVEEGYTAIYDGTLASLQEGWRMAGPGGFGRQEDCSIRGAGGMGLLWYDQELGENYSLKLDWKLTKDDNGGVFVGFPNPGDDPWVAVNKGYEIQIDATDADDRTTGAVYTFQGADEAARDAALKPVGQWNAYDIRVEGDRIRIYLNDVLVNDFTSTDPARLVNSFVGIQNHGSGEMVNYRNIRFKELTDEPVEELAISTTVQTRCLAGKVYVAVRATNDDTVPADITLTTPFGTKTVTGVQPGASAYQSFASRSTSVEAGTAQVSATGGDLTFQADVAYEAASCG

SEQ ID NO: 12 - protein, 1069 aa, Cellulosimicrobium cellulans IFO 15516
  MISC_FEATURE: amino acid sequence of aldehyde oxidase
DLPEQEPGVTLRTFQLAQNPGAVCTLKSGQTPNVDKLMPTIDWSTAEQFGAEDNFISQVSANLHVPADGQYQFRVTNDDGALVYIDGQLVVENDGPNDSTSVEGSATLTAGVHDLRVDYYEGSDKQRLTLAWKTPGSSTFEVIPTSALSTEAGVVRVTAPGYKYCEGATDTAGDGLRLDSVNPNYDLVDLRPEGFEPKVSGLAFTPDEKLAVVTTGEVSSGGWRPDPVSGEVYFLDGVLDADGPEDVTATKVADELLNPMGIEVVEDSIFVSERYQLTQLTDPDGDGFYDTHTKIAEWPDGGNFHEFAFGLIHDEDYFYVNLSVAINNGGATTNPQPAANRGTSIKIDRETGEVTYVAGGLRTPNGIGFGPEDEIFATDNQGAWLPSNKLIHIQQDKFYNHYTNPAGPFDANPVTPPAVWLPQNEIANSPGNPILVEDGEFAGQMLLGDVTYGGIQRAFLEKVDGEFQGAVFRHTAGLEVGVNRVIYGPDGALYAGGTGEGGNWGESGKLRYGLQKLVPVNEDSFDMKEMRVVEGGFEIEYTDPVSDEVVEKLADAYQVKQWRYVPTQQYGGPKVDEEPLFVTDATVSEDRTTVTLTIDGLKPDHVVYIRSPRPFASAEGTELLSTEAWYTLNSLPGYVAPADRGWYEAELAQPLGSSSIGSDHSNYSGSGFAAGMTNVGAGRTFSVTVPEAGTYPVNVRYANGIHPYTSLRSKNVSLHVNGQDLGQWNFPTTGSWKDWGVQTRDLQLQAGTNTITLAYEAGDEGNINIDVLSIGENPDICSPGEVEEGYTAIYDGTLASLQEGWRMAGPGGFGRQEDCSIRGAGGMGLLWYDQELGENYSLKLDWKLTKDDNGGVFVGFPNPGDDPWVAVNKGYEIQIDATDADDRTTGAVYTFQGADEAARDAALKPVGQWNAYDIRVEGDRIRIYLNDVLVNDFTSTDPARLVNSFVGIQNHGSGEMVNYRNIRFKELTDEPVEELAISTTVQTRCLAGKVYVAVRATNDDTVPADITLTTPFGTKTVTGVQPGASAYQSFASRSTSVEAGTAQVSATGGDLTFQADVAYEAASCG

SEQ ID NO: 13 - DNA, 3324 bp, Cellulosimicrobium cellulans IFO 15516
  misc_feature: DNA sequence of aldehyde oxidase containing signal peptide
atgctcgaacgcaccagcctgccggtgtcccgccggatcaggtaccgacgcggcgcggccgccctgacccttggcgccgtggtcgtcgcgggctgggccgtccccgccgcggccgacctccccgagcaggagcccggcgtcacgctgcggaccttccagctcgcgcagaaccccggggcggtctgcaccctgaagtccggccagacccccaacgtcgacaagctcatgccgaccatcgactggtcgacggccgagcagttcggcgcggaggacaacttcatctcgcaggtgagcgcgaacctgcacgtccccgcggacggccagtaccagttccgcgtgaccaacgacgacggcgcgctcgtctacatcgacggccagctcgtggtcgagaacgacggcccgaacgactcgacctccgtcgagggcagcgcgacgctcaccgcgggcgtgcacgacctgcgggtcgactactacgagggcagcgacaagcagcgcctcacgctcgcgtggaagacgccgggcagctcgacgttcgaggtgatcccgacgtcggcgctcagcaccgaggcgggcgtcgtgcgcgtgaccgcgcccggctacaagtactgcgagggcgcgaccgacacggccggcgacggcctgcgtctcgacagcgtcaaccccaactacgacctcgtcgacctgcgccccgagggcttcgagcccaaggtctcgggcctggccttcacgccggacgagaagctcgccgtcgtgacgaccggcgaggtcagctccggcgggtggcgccccgacccggtgtccggcgaggtgtacttcctcgacggcgtcctggacgcggacggccccgaggacgtcacggcgacgaaggtcgcggacgagctgctcaacccgatgggcatcgaggtcgtcgaggactcgatcttcgtctcggagcggtaccagctcacgcagctcaccgaccctgacggcgacggcttctacgacacgcacacgaagatcgcggagtggcccgacggcggcaacttccacgagttcgcgttcggcctgatccacgacgaggactacttctacgtcaacctctccgtggccatcaacaacggcggcgcgacgaccaacccgcagccggcggccaaccgcggcacgtcgatcaagatcgaccgcgagacgggtgaggtcacgtacgtcgcgggcggtctccgcacgccgaacggcatcggcttcggccccgaggacgagatcttcgcgacggacaaccagggtgcctggctcccgtcgaacaagctcatccacatccagcaggacaagttctacaaccactacacgaacccggccgggccgttcgacgcgaaccccgtcaccccgccggccgtgtggctcccgcagaacgagatcgccaactccccgggcaacccgatcctcgtcgaggacggcgagttcgccggccagatgctgctcggcgacgtgacctacggcggcatccagcgcgcgttcctcgagaaggtcgacggcgagttccagggtgcggtcttccgccacaccgcgggcctcgaggtcggcgtgaaccgtgtcatctacggtcccgacggcgcgctctacgccggcggcaccggtgagggcggcaactggggcgagtccggcaagctccggtacggcctccagaagctcgtccccgtgaacgaggactccttcgacatgaaggagatgcgcgtggtcgagggcggcttcgagatcgagtacaccgacccggtgtccgacgaggtcgtcgagaagctggccgacgcgtaccaggtcaagcagtggcgctacgtgccgacgcagcagtacggcggcccgaaggtcgacgaggagcccctcttcgtcaccgacgccaccgtgtccgaggaccgcacgaccgtcacgctgacgatcgacggcctcaagcccgaccacgtcgtctacatccgctcgccgcgcccgttcgcgtccgccgagggcaccgagctcctcagcaccgaggcctggtacacgctcaactcgctgcccggctacgtcgccccggccgaccgcggctggtacgaggcggagctggcccagccgctcggcagctcgagcatcggctcggaccacagcaactactccggttcgggcttcgccgcgggcatgacgaacgtcggtgccggccgcacgttctccgtgaccgtccccgaggcgggcacctacccggtcaacgtgcgctacgccaacggcatccacccgtacacgtcgctgcggtcgaagaacgtctcgctccacgtgaacggccaggacctgggccagtggaacttccccaccacgggaagctggaaggactggggcgtgcagacgcgcgacctccagctccaggccggcacgaacaccatcacgctcgcgtacgaggccggtgacgagggcaacatcaacatcgacgtcctgtcgatcggcgagaacccggacatctgctccccgggcgaggtcgaggagggctacaccgcgatctacgacggcaccctcgcgagcctccaggagggctggcgcatggccggtccgggtggcttcggccgccaggaggactgctcgatccgcggtgcgggtggcatgggcctgctctggtacgaccaggagctcggcgagaactacagcctgaagctcgactggaagctcacgaaggacgacaacggcggcgtgttcgtcgggttcccgaacccgggcgacgacccgtgggtggccgtcaacaagggctacgagatccagatcgacgcgacggacgccgacgaccgcaccaccggcgcggtctacacgttccagggcgccgacgaggcagcgcgcgacgccgccctcaagccggtcgggcagtggaacgcgtacgacatccgtgtcgagggagaccgcatccgcatctacctcaacgacgtgctcgtcaacgacttcacgagcaccgacccggcccggctggtgaacagcttcgtcggcatccagaaccacggcagcggcgagatggtcaactaccggaacatccggttcaaggagctgaccgacgagccggtcgaggagctcgcgatctcgacgacggtccagacccgctgcctggcgggcaaggtctacgtcgcggtccgtgccacgaacgacgacacggtgccggcggacatcacgctgacgacgccgttcgggacgaagacggtgaccggcgtccagccgggtgcctccgcgtaccagtcgttcgcgtcgcgctcgacctcggtcgaggcgggcaccgcccaggtgtccgcgacgggcggcgacctgacgttccaggcggacgtcgcctacgaggccgcgtcctgcggctga

SEQ ID NO: 14 - DNA, 3210 bp, Cellulosimicrobium cellulans IFO 15516
  misc_feature: DNA sequence of aldehyde oxidase
gacctccccgagcaggagcccggcgtcacgctgcggaccttccagctcgcgcagaaccccggggcggtctgcaccctgaagtccggccagacccccaacgtcgacaagctcatgccgaccatcgactggtcgacggccgagcagttcggcgcggaggacaacttcatctcgcaggtgagcgcgaacctgcacgtccccgcggacggccagtaccagttccgcgtgaccaacgacgacggcgcgctcgtctacatcgacggccagctcgtggtcgagaacgacggcccgaacgactcgacctccgtcgagggcagcgcgacgctcaccgcgggcgtgcacgacctgcgggtcgactactacgagggcagcgacaagcagcgcctcacgctcgcgtggaagacgccgggcagctcgacgttcgaggtgatcccgacgtcggcgctcagcaccgaggcgggcgtcgtgcgcgtgaccgcgcccggctacaagtactgcgagggcgcgaccgacacggccggcgacggcctgcgtctcgacagcgtcaaccccaactacgacctcgtcgacctgcgccccgagggcttcgagcccaaggtctcgggcctggccttcacgccggacgagaagctcgccgtcgtgacgaccggcgaggtcagctccggcgggtggcgccccgacccggtgtccggcgaggtgtacttcctcgacggcgtcctggacgcggacggccccgaggacgtcacggcgacgaaggtcgcggacgagctgctcaacccgatgggcatcgaggtcgtcgaggactcgatcttcgtctcggagcggtaccagctcacgcagctcaccgaccctgacggcgacggcttctacgacacgcacacgaagatcgcggagtggcccgacggcggcaacttccacgagttcgcgttcggcctgatccacgacgaggactacttctacgtcaacctctccgtggccatcaacaacggcggcgcgacgaccaacccgcagccggcggccaaccgcggcacgtcgatcaagatcgaccgcgagacgggtgaggtcacgtacgtcgcgggcggtctccgcacgccgaacggcatcggcttcggccccgaggacgagatcttcgcgacggacaaccagggtgcctggctcccgtcgaacaagctcatccacatccagcaggacaagttctacaaccactacacgaacccggccgggccgttcgacgcgaaccccgtcaccccgccggccgtgtggctcccgcagaacgagatcgccaactccccgggcaacccgatcctcgtcgaggacggcgagttcgccggccagatgctgctcggcgacgtgacctacggcggcatccagcgcgcgttcctcgagaaggtcgacggcgagttccagggtgcggtcttccgccacaccgcgggcctcgaggtcggcgtgaaccgtgtcatctacggtcccgacggcgcgctctacgccggcggcaccggtgagggcggcaactggggcgagtccggcaagctccggtacggcctccagaagctcgtccccgtgaacgaggactccttcgacatgaaggagatgcgcgtggtcgagggcggcttcgagatcgagtacaccgacccggtgtccgacgaggtcgtcgagaagctggccgacgcgtaccaggtcaagcagtggcgctacgtgccgacgcagcagtacggcggcccgaaggtcgacgaggagcccctcttcgtcaccgacgccaccgtgtccgaggaccgcacgaccgtcacgctgacgatcgacggcctcaagcccgaccacgtcgtctacatccgctcgccgcgcccgttcgcgtccgccgagggcaccgagctcctcagcaccgaggcctggtacacgctcaactcgctgcccggctacgtcgccccggccgaccgcggctggtacgaggcggagctggcccagccgctcggcagctcgagcatcggctcggaccacagcaactactccggttcgggcttcgccgcgggcatgacgaacgtcggtgccggccgcacgttctccgtgaccgtccccgaggcgggcacctacccggtcaacgtgcgctacgccaacggcatccacccgtacacgtcgctgcggtcgaagaacgtctcgctccacgtgaacggccaggacctgggccagtggaacttccccaccacgggaagctggaaggactggggcgtgcagacgcgcgacctccagctccaggccggcacgaacaccatcacgctcgcgtacgaggccggtgacgagggcaacatcaacatcgacgtcctgtcgatcggcgagaacccggacatctgctccccgggcgaggtcgaggagggctacaccgcgatctacgacggcaccctcgcgagcctccaggagggctggcgcatggccggtccgggtggcttcggccgccaggaggactgctcgatccgcggtgcgggtggcatgggcctgctctggtacgaccaggagctcggcgagaactacagcctgaagctcgactggaagctcacgaaggacgacaacggcggcgtgttcgtcgggttcccgaacccgggcgacgacccgtgggtggccgtcaacaagggctacgagatccagatcgacgcgacggacgccgacgaccgcaccaccggcgcggtctacacgttccagggcgccgacgaggcagcgcgcgacgccgccctcaagccggtcgggcagtggaacgcgtacgacatccgtgtcgagggagaccgcatccgcatctacctcaacgacgtgctcgtcaacgacttcacgagcaccgacccggcccggctggtgaacagcttcgtcggcatccagaaccacggcagcggcgagatggtcaactaccggaacatccggttcaaggagctgaccgacgagccggtcgaggagctcgcgatctcgacgacggtccagacccgctgcctggcgggcaaggtctacgtcgcggtccgtgccacgaacgacgacacggtgccggcggacatcacgctgacgacgccgttcgggacgaagacggtgaccggcgtccagccgggtgcctccgcgtaccagtcgttcgcgtcgcgctcgacctcggtcgaggcgggcaccgcccaggtgtccgcgacgggcggcgacctgacgttccaggcggacgtcgcctacgaggccgcgtcctgcggctga

SEQ ID NO: 15 - protein, 27 aa, Streptomyces sp. KNK269
  MISC_FEATURE: N-terminal amino acid sequence of subunit gamma of aldehyde oxidase
GADLEPEPVVDEHSTVTLNVNGDPTTL

SEQ ID NO: 16 - protein, 40 aa, Streptomyces sp. KNK269
  MISC_FEATURE: N-terminal amino acid sequence of subunit beta of aldehyde oxidase
MKPFGYVRPASPDEAVRLCAEGSGARFLGGGTNLVDLMKL

SEQ ID NO: 17 - protein, 36 aa, Streptomyces sp. KNK269
  MISC_FEATURE: amino acid sequence (from Ala231 to Ala266) of subunit beta of aldehyde oxidase
ARDRASFAFALVSVAATLRVDAGRVEHATLAFGGVA

SEQ ID NO: 18 - protein, 24 aa, Streptomyces sp. KNK269
  MISC_FEATURE: N-terminal amino acid sequence of subunit alpha of aldehyde oxidase
APQFPGAPVVRREARDKVTGTARY

SEQ ID NO: 19 - protein, 38 aa, Streptomyces sp. KNK269
  MISC_FEATURE: amino acid sequence (from Leu261to Glu298) of subunit alpha of aldehyde oxidase
LVFPRAQLAEVVGHRAPTIQRVRLGADLDGVLTAVSHE

SEQ ID NO: 20 - protein, 35 aa, Streptomyces sp. KNK269
  MISC_FEATURE: amino acid sequence (from Gly659 to Thr693) of subunit alpha of aldehyde oxidase
GIGEIGIVGTAAAIGNAVRHATGARLRELPLTTDT

SEQ ID NO: 21 - DNA, 29 bp, Artificial Sequence
  mixed DNA primer (1) corresponding to N-terminal amino acid sequence of subunit gamma of aldehyde oxidase of Streptomyces sp. KNK269
gacctsgarccsgarccsgtsgtsgacga

SEQ ID NO: 22 - DNA, 23 bp, Artificial Sequence
  complementary mixed DNA primer (2) corresponding to amino acid sequence (from Asp251 to Ala258) of subunit beta of Streptomyces sp. KNK269 aldehyde oxidase
gcgtgytcsacscgbccsgcgtc

SEQ ID NO: 23 - DNA, 23 bp, Artificial Sequence
  mixed DNA primer (3) corresponding to amino acid sequence (from Asp251 to Ala258) of subunit beta of Streptomyces sp. KNK269 aldehyde oxidase
gacgcsggvcgsgtsgarcacgc

SEQ ID NO: 24 - DNA, 23 bp, Artificial Sequence
  complementary mixed DNA primer (4) corresponding to amino acid sequence (from Leu274 to Glu281) of subunit alpha of Streptomyces sp. KNK269 aldehyde oxidase
cgctggatsgtsggsgcscggtg

SEQ ID NO: 25 - DNA, 23 bp, Artificial Sequence
  mixed DNA primer (5) corresponding to amino acid sequence (from Leu274 to Glu281) of subunit alpha of Streptomyces sp. KNK269 aldehyde oxidase
caccgsgcsccsacsatccagcg

SEQ ID NO: 26 - DNA, 23 bp, Artificial Sequence
  complementary mixed DNA primer (6) corresponding to amino acid sequence (from Leu686 to Glu693) of subunit alpha of Streptomyces sp. KNK269 aldehyde oxidase
gtgtcsgtsgtsagsggsagytc

SEQ ID NO: 27 - protein, 10 aa, Microbacterium sp. KNK011
  MISC_FEATURE: N-terminal amino acid sequence of aldehyde oxidase
VNTPADLPKQ

SEQ ID NO: 28 - protein, 34 aa, Microbacterium sp. KNK011
  MISC_FEATURE: internal amino acid sequence of aldehyde oxidase
VDGEFQGAVFRHSAGFTVGVNRVIEGPDTSLYIG

SEQ ID NO: 29 - protein, 19 aa, Microbacterium sp. KNK011
  MISC_FEATURE: internal amino acid sequence of aldehyde oxidase
IFLNGTLVNDFTSVDPARE

SEQ ID NO: 30 - DNA, 29 bp, Artificial Sequence
  mixed DNA primer (1) corresponding to N-terminal amino acid sequence of aldehydeoxidase from Microbacterium sp. KNK011
  misc_feature (15)..(15): n is a, c, g, or t
gtcaacacsccsgcngayctbccsaarca

SEQ ID NO: 31 - DNA, 26 bp, Artificial Sequence
  complementary mixed DNA primer (2) corresponding to amino acid sequence (from Asp513 to Phe521) of aldehyde oxidase from Microbacterium sp. KNK011
  misc_feature (21)..(21): n is a, c, g, or t
aasacbgcsccctgraaytcnccrtc

SEQ ID NO: 32 - DNA, 26 bp, Artificial Sequence
  mixed DNA primer (3) corresponding to amino acid sequence (from Asp513 to Phe521) of aldehydeoxidase from Microbacterium sp. KNK011
  misc_feature (21)..(21): n is a, c, g, or t
  misc_feature (24)..(24): n is a, c, g, or t
gacggsgarttccagggvgcngtntt

SEQ ID NO: 33 - DNA, 32 bp, Artificial Sequence
  complementary mixed DNA primer (4) corresponding to amino acid sequence (from Phe959 to Thr969) of aldehyde oxidase from Microbacterium sp. KNK011
  misc_feature (27)..(27): n is a, c, g, or t
gtgaagtcgttsacsagsgtgccrttnagraa

SEQ ID NO: 34 - DNA, 32 bp, Artificial Sequence
  DNA primer containing Nde I restriction site for cloning of aldehyde oxidase from Microbacterium sp. KNK011
gtgccgccgtcgcccatatggtcaacaccccg

SEQ ID NO: 35 - DNA, 29 bp, Artificial Sequence
  DNA primer containing EcoR I restriction site for cloning of aldehyde oxidase from Microbacterium sp. KNK011
ggagagcatggggaattctgggacatgag

SEQ ID NO: 36 - protein, 23 aa, Cellulosimicrobium cellulans IFO 15516
  MISC_FEATURE: N-terminal amino acid sequence of aldehyde oxidase
DLPEQEPGVTLRTFQLAQNPTAV

SEQ ID NO: 37 - protein, 31 aa, Cellulosimicrobium cellulans IFO 15516
  MISC_FEATURE: internal amino acid sequence of aldehyde oxidase
KTPGSTVFEVIPTSALSTEAGVVYVTAPGVK

SEQ ID NO: 38 - protein, 34 aa, Cellulosimicrobium cellulans IFO 15516
  MISC_FEATURE: internal amino acid sequence of aldehyde oxidase
KIAEWPDGGNFHEIAFELIHDEDYFYVNLSVAIN

SEQ ID NO: 39 - DNA, 25 bp, Artificial Sequence
  mixed DNA primer (1) corresponding to N-terminal amino acid sequence of aldehydeoxidase from Cellulosimicrobium cellulans IFO 15516
acctsccsgagcaggagccsggsgt

SEQ ID NO: 40 - DNA, 26 bp, Artificial Sequence
  complementary mixed DNA primer (2) corresponding to amino acid sequence (from Ile350 to Val358) of aldehyde oxidase from Cellulosimicrobium cellulans IFO 15516
acgtagaagtagtcctcgtcgtggat

SEQ ID NO: 41 - DNA, 23 bp, Artificial Sequence
  mixed DNA primer (3) corresponding to amino acid sequence (from Thr176 to Thr183) of aldehydeoxidase from Cellulosimicrobium cellulans IFO 15516
acsgtsttcgaggtsatcccsac

SEQ ID NO: 42 - DNA, 25 bp, Artificial Sequence
  complementary DNA primer (4) corresponding to DNA sequence (2521-2545) of aldehyde oxidase from MIicrobacterium sp. KNK011
tcaggctctcgagcgtgccgtcgaa

(19) United States
(12) Patent Application Publication
(10) Pub. No.: US 2007/0026510 A1
(43) Pub. Date: Feb. 1, 2007

(54) Process for biochemical production of glyoxylic acid

(75) Inventors: Akira Iwasaki, Hyogo (JP); Takehiro Matsumoto, Hyogo (JP); Motohisa Washida, Hyogo (JP); Hiroshi Watanabe, Hyogo (JP); Junzou Hasegawa, Hyogo (JP); Sakayu Shimizu, Kyoto (JP)

Correspondence Address: SUGHRUE MION, PLLC, 2100 PENNSYLVANIA AVENUE, N.W., SUITE 800, WASHINGTON, DC, 20037, US

(73) Assignee: Kaneka Corporation, Osaka (JP)

(21) Appl. No.: 10/544,618

(22) PCT Filed: Feb. 13, 2004
(86) PCT No.: PCT/JP04/01577

§ 371 Date: Jun. 21, 2006

(30) Foreign Application Priority Data
Feb. 14, 2003 (JP) 2003-036303
Sep. 26, 2003 (JP) 2003-336234

(51) Int. Cl. C12P 7/50; C12N 1/20

(52) U.S. Cl. 435/143; 435/252.34

(57) ABSTRACT

The present invention provides an industrially advantageous process for biochemical production of glyoxylic acid from glyoxal. More specifically, the present invention provides a process for production of glyoxylic acid, which is characterized in that the process comprises allowing oxidoreductase that can convert glyoxal into glyoxylic acid, such as oxidase and dehydrogenase, to act on glyoxal, so as to convert glyoxal into glyoxylic acid.

### TECHNICAL FIELD

The present invention relates to a process for producing glyoxylic acid from glyoxal using microorganisms and/or enzymes derived from microorganisms. Glyoxylic acid is used as a synthetic raw material for vanillin, ethyl vanillin, or the like. This compound is useful as an intermediate for synthesizing agricultural chemicals and pharmaceuticals.

### BACKGROUND ART

As a process for producing glyoxylic acid, chemical methods such as nitric acid oxidation of glyoxal has conventionally been known. At present, almost all of glyoxylic acids are produced by such chemical methods. However, chemical methods such as nitric acid oxidation of glyoxal are likely to generate by-products such as organic acids other than glyoxylic acid. Such by-products affect the quality of the produced glyoxylic acid. In order to remove these by-products, complicated steps are required. In addition, the treatment of a large amount of salt waste, which is produced during the neutralization step of a large amount of nitric acid used, has been problematic.

Examples of a process for biochemical production of glyoxylic acid may include: a process for converting glycolic acid into glyoxylic acid using glycolate oxidase derived from plants (see National Publication of International Patent Application Nos. 7-502895 and 8-508159); and a process for converting glycolic acid into glyoxylic acid using microorganisms (see Japanese Patent Laid-Open Nos. 7-163380 and 8-322581). However, a process for biochemical synthesis of glyoxylic acid from glyoxal, which is an inexpensively available compound that is easily synthesized from ethylene glycol or acetaldehyde and which is used as a material in the chemical synthetic method of glyoxylic acid, has not yet been reported.

Moreover, it has been confirmed that oxidase that oxidizes an aldehyde group exists in animals, plants, or the like. However, it has not been reported that such an enzyme exhibits activity to glyoxal. Furthermore, it has been known that several types of white-rot fungi such as Phanerochaete chrysosporium produce an enzyme having activity of reacting with glyoxal to generate hydrogen peroxide (which is called glyoxal oxidase) to outside of the microbial cells (hereinafter referred to briefly as “cells”), thereof (see Journal of Bacteriology, (1987), 169, 2195-2201; and Pro. Natl. Acad. Sci. (1990), 87, 2936-2940). However, a product produced from glyoxal during the oxidization reaction of the glyoxal with the enzyme that is produced by the above white-rot fungi has not yet been identified. Since the enzyme derived from these wood-rotting fungi has oxidization activity to glyoxylic acid, which is the same level as that to glyoxal, it is difficult that glyoxal is converted into glyoxylic acid and it is then accumulated, using the above enzyme. Other than the enzyme derived from the wood-rotting fungi, no oxidase derived from microorganisms that oxidizes glyoxal has been reported.

Thus, it is an object of the present invention to provide a microorganism having activity of converting glyoxal into glyoxylic acid and/or an enzyme having the above activity, and a process for efficient production of glyoxylic acid using these items.

### DISCLOSURE OF THE INVENTION

As a result of intensive studies directed towards developing a process for efficient production of glyoxylic acid, the present inventors have found a microorganism having activity of converting glyoxal into glyoxylic acid. The present inventors have studied in detail the synthesis of glyoxylic acid using such a microorganism and/or a solution that contains enzyme obtained from the above microorganism, thereby completing the present invention.

That is to say, the present invention relates to a process for production of glyoxylic acid, which is characterized in that the process comprises allowing oxidoreductase that can convert glyoxal into glyoxylic acid, or at least one or a mixture consisting of two or more types selected from the group consisting of a culture broth, a supernatant of the culture broth, cells, and a processed product of a microorganism that can produce the above described oxidoreductase, to act on glyoxal, so as to convert glyoxal into glyoxylic acid.

The above-described oxidoreductase is preferably oxidase.

The above-described oxidoreductase is preferably an enzyme obtained from at least one microorganism selected from the group consisting of the genus Stenotrophomonas, Streptomyces, Pseudomonas, Microbacterium, Achromobacter, Cellulomonas, Cellulosimicrobium, and Morganella.

The above described microorganism is preferably Stenotrophomonas sp. KNK235 (deposit institution: National Institute of Advanced Industrial Science and Technology; address: AIST Tsukuba, Central 6, Higashi 1-1-1, Tsukuba, Ibaraki, Japan (postal code: 305-8566); deposit date: Sep. 6, 2002; accession No. FERM P-19002), Streptomyces sp. KNK269 (deposit institution: National Institute of Advanced Industrial Science and Technology; address: AIST Tsukuba, Central 6, Higashi 1-1-1, Tsukuba, Ibaraki, Japan (postal code: 305-8566); deposit date: Sep. 6, 2002; accession No. FERMBP-08556), Pseudomonas sp. KNK058 (deposit institution: National Institute of Advanced Industrial Science and Technology; address: AIST Tsukuba, Central 6, Higashi 1-1-1, Tsukuba, Ibaraki, Japan (postal code: 305-8566); deposit date: Dec. 13, 2002; accession No. FERM BP-08555), Pseudomonas sp. KNK254 (deposit institution: National Institute of Advanced Industrial Science and Technology; address: AIST Tsukuba, Central 6, Higashi 1-1-1, Tsukuba, Ibaraki, Japan (postal code: 305-8566); deposit date: Sep. 6, 2002; accession No. FERM P-19003), Microbacterium sp. KNK011 (deposit institution: National Institute of Advanced Industrial Science and Technology; address: AIST Tsukuba, Central 6, Higashi 1-1-1, Tsukuba, Ibaraki, Japan (postal code: 305-8566); deposit date: Dec. 13, 2002; accession No. FERM BP-08554), Achromobacter sp. IFO 13495 (deposit institution: National Institute of Technology and Evaluation, Biological Resource Center (NBRC); address: Kazusa Kamatari 2-5-8, Kisarazu, Chiba, Japan (postal code: 292-0818)), Cellulomonas sp. JCM 2471 (deposit institution: Riken Bioresource Center, Japan Collection of Microorganisms (JCM); address: Hirosawa 2-1, Wako, Saitama, Japan (postal code: 351-0198)), Cellulomonas turbata IFO 15012 (deposit institution: National Institute of Technology and Evaluation, Biological Resource Center (NBRC); address: Kazusa Kamatari 2-5-8, Kisarazu, Chiba, Japan (postal code: 292-0818)), Cellulomonas turbata IFO 15014 (deposit institution: National Institute of Technology and Evaluation, Biological Resource Center (NBRC); address: Kazusa Kamatari 2-5-8, Kisarazu, Chiba, Japan (postal code: 292-0818)), Cellulomonas turbata IFO 15015 (deposit institution: National Institute of Technology and Evaluation, Biological Resource Center (NBRC); address: Kazusa Kamatari 2-5-8, Kisarazu, Chiba, Japan (postal code: 292-0818)), Cellulosimicrobium cellulans IFO 15013 (deposit institution: National Institute of Technology and Evaluation, Biological Resource Center (NBRC); address: Kazusa Kamatari 2-5-8, Kisarazu, Chiba, Japan (postal code: 292-0818)), Cellulosimicrobium cellulans IFO 15516 (deposit institution: National Institute of Technology and Evaluation, Biological Resource Center (NBRC); address: Kazusa Kamatari 2-5-8, Kisarazu, Chiba, Japan (postal code: 292-0818)), Cellulosimicrobium cellulans JCM 6201 (deposit institution: Riken Bioresource Center, Japan Collection of Microorganisms (JCM); address: Hirosawa 2-1, Wako, Saitama, Japan (postal code: 351-0198)), or Morganella morganii IFO 3848 (deposit institution: National Institute of Technology and Evaluation, Biological Resource Center (NBRC); address: Kazusa Kamatari 2-5-8, Kisarazu, Chiba, Japan (postal code: 292-0818)).

In the above-described process for production of glyoxylic acid, catalase is preferably allowed to coexist during the reaction.

In addition, the present invention relates to an aldehyde oxidase derived from a microorganism that acts on glyoxal to generate glyoxylic acid.

The activity of the above described aldehyde oxidase to glyoxylic acid is preferably one-tenth or less of the above activity to glyoxal.

The above described aldehyde oxidase is preferably produced by at least one microorganism selected from the group consisting of the genus Stenotrophomonas, Streptomyces Pseudomonas, Microbacterium, Achromobacter, Cellulomonas, Cellulosimicrobium, and Morganella.

The above described microorganism is preferably Stenotrophomonas sp. KNK235 (FERM P-19002), Streptomyces sp. KNK269 (FERM BP-08556), Pseudomonas sp. KNK058 (FERM BP-08555), Pseudomonas sp. KNK254 (FERM P-19003), Microbacterium sp. KNK011 (FERM BP-08554), Achromobacter sp. IFO 13495, Cellulomonas sp. JCM 2471, Cellulomonas turbata IFO 15012, Cellulomonas turbata IFO 15014, Cellulomonas turbata IFO 15015, Cellulosimicrobium cellulans IFO 15013, Cellulosimicrobium cellulans IFO 15516, Cellulosimicrobium cellulans JCM 6201, or Morganella morganii IFO 3848.

The above described aldehyde oxidase is preferably produced by a microorganism belonging to the genus Streptomyces and preferably has the following physicochemical properties (1) to (3):
(1) optimum pH: 6 to 9;
(2) heat stability: the aldehyde oxidase retains activity of 90% or more after it has been treated at pH 7.2 at 60° C. for 20 minutes; and
(3) molecular weight: the aldehyde oxidase has a molecular weight of approximately 110,000 in gel filtration analysis, and has three subunit proteins with molecular weights of approximately 25,000, approximately 35,000, and approximately 80,000 in SDS-polyacrylamide gel electrophoresis analysis.

The above described aldehyde oxidase is preferably produced by a microorganism belonging to the genus Pseudomonas and preferably has the following physicochemical properties (1) to (3):
(1) molecular weight: approximately 150,000 in gel filtration analysis;
(2) optimum reaction temperature: 60° C. to 70° C.; and
(3) optimum reaction pH: 5 to 7.

The above described aldehyde oxidase is preferably generated by a microorganism belonging to the genus Microbacterium inside and outside of the cells thereof, and preferably has the following physicochemical properties: molecular weight: a single protein has a molecular weight of approximately 110,000 in SDS-polyacrylamide gel electrophoresis analysis.

The above described aldehyde oxidase is produced by a microorganism belonging to the genus Cellulosimicrobium inside and outside of the cells thereof, and preferably has the following physicochemical properties: molecular weight: a single protein has a molecular weight of approximately 90,000 to 100,000 in SDS-polyacrylamide gel electrophoresis analysis.

The above-described microorganism belonging to the genus Streptomyces is preferably Streptomyces sp. KNK269 (FERM BP-08556).

The above-described microorganism belonging to the genus Pseudomonas is preferably Pseudomonas sp. KNK058 (FERM BP-08555).

The above-described microorganism belonging to the genus Microbacterium is preferably Microbacterium sp. KNK011 (FERM BP-08554).

The above-described microorganism belonging to the genus Cellulosimicrobium is preferably Cellulosimicrobium cellulans IFO 15516.

The above described aldehyde oxidase preferably has a protein described in the following (a) or (b) as a subunit:
(a) a protein having an amino acid sequence represented by SEQ ID NO: 1, 2, or 3; or
(b) a protein comprising an amino acid sequence resulting from deletion, substitution, or addition of one or several amino acids in the amino acid sequence (a).

The above described aldehyde oxidase preferably has a protein encoded by the DNA described in the following (a) or
(b) as a subunit:
(a) DNA having a nucleotide sequence represented by SEQ ID NO: 4, 5, or 6; or
(b) DNA which hybridizes with any one DNA consisting of a nucleotide sequence that is complementary to the DNA consisting of the nucleotide sequence (a) under stringent conditions.

The above described aldehyde oxidase preferably has the amino acid sequence described in the following (a) or (b):
(a) an amino acid sequence represented by SEQ ID NO: 7, 8, 11, or 12; or
(b) an amino acid sequence resulting from deletion, substitution, or addition of one or several amino acids in the amino acid sequence (a).

The above described aldehyde oxidase is preferably encoded by the DNA described in the following (a) or (b):
(a) DNA consisting of a nucleotide sequence represented by SEQ ID NO: 9, 10, 13, or 14; or
(b) DNA which hybridizes with DNA consisting of a nucleotide sequence that is complementary to the DNA consisting of the nucleotide sequence (a) under stringent conditions.

Moreover, the present invention also relates to DNA encoding the above described aldehyde oxidase.

Specifically, the above described DNA is preferably DNA encoding a subunit of the above described aldehyde oxidase, which comprises the DNA described in the following (a) or (b):
(a) DNA having a nucleotide sequence represented by SEQ ID NO: 4, 5, or 6; or
(b) DNA which hybridizes with any one DNA consisting of a nucleotide sequence that is complementary to the DNA consisting of the nucleotide sequence is (a) under stringent conditions.

The above described DNA is preferably DNA encoding the above described aldehyde oxidase, which comprises the DNA described in the following (a) or (b):
(a) DNA having a nucleotide sequence represented by SEQ ID NO: 9, 10, 13, or 14; or
(b) DNA which hybridizes with any one DNA consisting of a nucleotide sequence that is complementary to the DNA consisting of the nucleotide sequence (a) under stringent conditions.

The above described DNA is preferably DNA encoding a subunit of the above described aldehyde oxidase, which comprises an acid sequence resulting from deletion, substitution, or addition of one or several amino acids in the amino acid sequence represented by SEQ ID NO: 1, 2, or 3.

The above described DNA is preferably DNA encoding the above described aldehyde oxidase, which comprises an amino acid sequence resulting from deletion, substitution, or addition of one or several amino acids in the amino acid sequence represented by SEQ ID NO: 7, 8, 11, or 12.

### BRIEF DESCRIPTION OF THE DRAWINGS

FIG. 1 is a view showing the principle of a process for measuring activity in an oxidase reaction.

FIG. 2 is a view showing the optimum reaction pH of the enzyme derived from the KNK269 strain of the present invention. A 0.1 M MacIlvine buffer(●, filled circle), a 0.1 M phosphate buffer(◯, open circle), a 0.1 MTricine buffer(Δ, triangle), or a 0.1 M Glycine-HCl buffer (X) was used as a buffer.

FIG. 3 is a view showing the heat stability of the enzyme derived from the KNK269 strain of the present invention.

FIG. 4 is a view showing the optimum reaction temperature of the enzyme derived from the KNK058 strain of the present invention.

FIG. 5 is a view showing the optimum reaction pH of the KNK058 strain of the present invention. A 0.1 M MacIlvine buffer (●, filled circle), a 0.1 M phosphate buffer (◯, open circle), or a 0.1 M Tricine buffer (x) was used as a buffer.

### BEST MODE FOR CARRYING OUT THE INVENTION

The conversion reaction of glyoxal into glyoxylic acid of the present invention is shown in the following formulae 1 and 2:

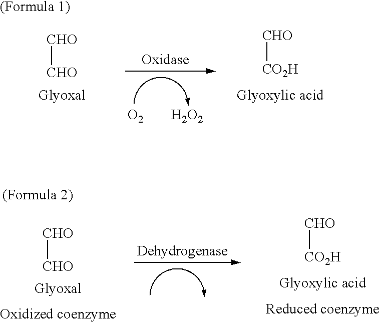

The reaction represented by the above formula 1 takes place when oxidase or a microorganism containing the oxidase intervenes. The reaction represented by the above formula 2 takes place when dehydrogenase or a microorganism containing the dehydrogenase intervenes. In the present specification, conversion of glyoxal into glyoxylic acid includes both the conversion due to the reaction represented by the above formula 1 and the conversion due to the reaction represented by the above formula 2. Accordingly, the term “oxidoreductase that can convert glyoxal into glyoxylic acid”is used to mean oxidase or dehydrogenase. Such oxidoreductase may be either oxidase or dehydrogenase, as long as it is an enzyme converting aldehyde into carboxylic acid. In terms of accumulation of glyoxylic acid, an enzyme that does not have activity or has only low activity to glyoxylic acid is particularly preferable.

Moreover, it is also possible to convert glyoxal into glyoxylic acid using at least one or a mixture consisting of two or more selected from the group consisting of a culture broth, a supernatant of the culture broth, cells, and a processed product of a microorganism that can produce the above oxidoreductase. When the reaction is catalyzed by dehydrogenase (formula 2), a coenzyme (for example, NAD (nicotinamide adenine dinucleotide) or NADP (nicotinamide adenine dinucleotide phosphate)) is necessary, as well as a substrate. On the other hand, when the reaction is catalyzed by oxidase (formula 1), if oxygen exists as well as glyoxal used as a substrate, the reaction progresses. Accordingly, the use of oxidase is advantageous in terms of cost reduction.

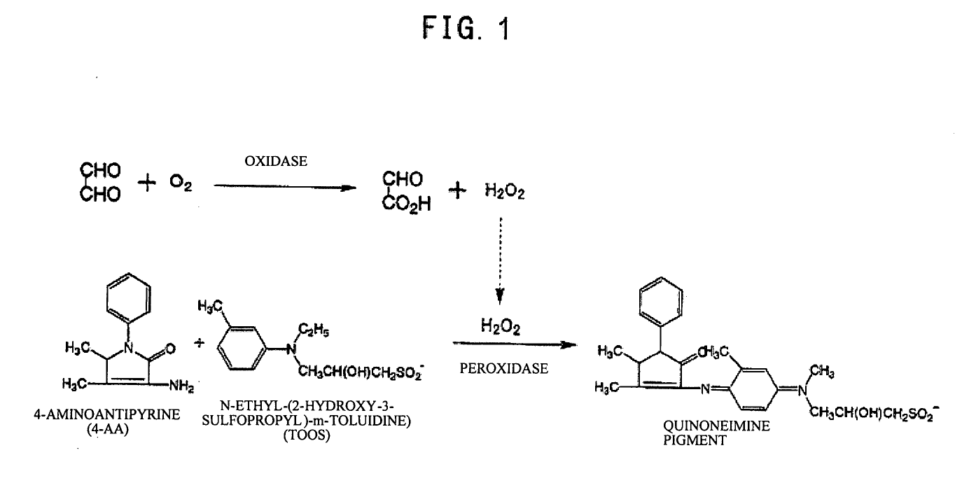

When the reaction is catalyzed by oxidase, not only glyoxylic acid but also hydrogen peroxide is produced. Oxidase activity of interest can easily be detected by detecting such hydrogen peroxide. As shown in FIG. 1, in the present invention, detection and quantification of the oxidase activity of interest can be carried out by allowing hydrogen peroxide produced as a result of an oxidization reaction to react with 4-aminoantipyrine (hereinafter referred to as 4-AA) and N-ethyl-(2-hydroxy-3-sulfopropyl)-m-toluidine (hereinafter referred to as TOOS), and then detecting and quantifying a quinoneimine pigment produced.

Specifically, 0.1 ml of cell suspension or enzyme solution is added to 0.9 ml of a 100 mM phosphate buffer (pH 7) having the composition indicated below, and an increase in the absorbance at a wavelength of 555 nm is measured at 30° C. In the present invention, enzyme activity for generating 1 μmol H₂O₂ per minute is defined as 1 unit.

(Composition)

| | | |
|---|---|---|
| Glyoxal | 20 | mM |
| 4-AA | 0.67 | mM |
| TOOS | 1.09 | mM |
| Peroxidase derived from horseradish | 2 | U/mL |
| (hereinafter referred to as POD) | | |

Quantification of glyoxal and glyoxylic acid can be carried out by high performance liquid chromatography. Analysis by high performance liquid chromatography can be carried out, for example, using a Bio-Rad Aminex HPX-87H column (7.8 mm×300 mm), also using a 5 mM H₂SO₄ aqueous solution as a solvent, at a flow rate of 0.4 ml/min. Detection is carried out by measuring absorbance at 230 nm or refractive index. Under the present conditions, glyoxal is eluted at 16 minutes, and glyoxylic acid is eluted at 15 minutes.

Microorganisms having oxidase activity of interest can be obtained by the following screening, for example. 0.2 ml of a supernatant obtained by suspending 2 g each of a soil sample collected from several regions in Japan in 10 ml of a saline solution was added to 5 ml of an S medium (pH 7) that had been sterilized by autoclaving (121° C., 20 minutes), which consisted of 10 g of glyoxal, ethylene glycol, propylene glycol, or glycolaldehyde used as a carbon source, 2 g of ammonium nitrate, 1 g of dipotassium hydrogen phosphate, 1 g of sodium dihydrogen phosphate, 0.1 g of yeast extract, 0.2 g of magnesium sulfate heptahydrate, and 0.1 g of calcium chloride dihydrate (all of which were amounts contained in 1 liter). It was subjected to enrichment culture at 28° C. for 3 to 7 days. 0.1 ml each of a culture broth, in which cells had grown, was spreaded onto S medium plate containing 2% agar. It was then inoculated at 28° C. for 3 to 7 days. Thereafter, growing colonies were subjected to static culture using S medium plate containing 2% agar medium again. Strains, the growth of which had been confirmed, were defined as assimilating strains for each carbon source. Thereafter, these assimilating strains were examined in terms of activity of converting glyoxal into glyoxylic acid. Each strain was subjected to culture in 5 ml of S medium placed in a test tube at 28° C. with reciprocal shaking for 3 to 5 days. Thereafter, cells were collected by centrifugation, were washed with a saline solution, and were then suspended in 0.5 ml of 100 mM Tris-HCl buffer (pH 8). 0.1 ml of the cell suspension was added to 0.2 ml of 100 mM Tris-HCl buffer (pH 8) containing 50 mM glyoxal, and the mixture was shaken at 28° C. for 6 to 12 hours. Thereafter, the reaction solution was centrifuged, and the obtained supernatant was analyzed by high performance liquid chromatography, thereby confirming and quantifying generation of glyoxylic acid.

When oxidization of glyoxal is catalyzed by the aforementioned oxidase, not only glyoxylic acid but also hydrogen peroxide is produced. Thus, oxidase-producing strains can be found by detecting hydrogen peroxide produced during the reaction. That is to say, 0.1 ml of cell suspension obtained by culture in the aforementioned S medium was added to 0.1 ml of 100 mM phosphate buffer containing 50 mM glyoxal, 1.34 mM 4-AA, 2.18 mM TOOS, and 4 U/ml peroxidase. The obtained mixture was shaken at 28° C. for 2 hours. Thereafter, reaction solutions, the color of which became violet, namely, strains generating hydrogen peroxide as a result of the reaction with glyoxal, were selected, so as to obtain strains having glyoxal oxidase activity.

Examples of microorganisms that can convert glyoxal into glyoxylic acid may include those belonging to the genus Stenotrophomonas, Streptomyces, Pseudomonas, Microbacterium, Achromobacter, Cellulomonas, Cellulosimicrobium, and Morganella. Of these, examples of typical microorganisms may include Stenotrophomonas sp. KNK235 (FERM P-19002), Streptomyces sp. KNK269 (FERM BP-08556), Pseudomonas sp. KNK058 (FERM BP-08555), Pseudomonas sp. KNK254 (FERM P-19003), Microbacterium sp. KNK011 (FERM BP-08554), Achromobacter sp. IFO 13495, Cellulomonas sp. JCM 2471, Cellulomonas turbata IFO 15012, Cellulomonas turbata IFO 15014, Cellulomonas turbata IFO 15015, Cellulosimicrobium cellulans IFO 15013, Cellulosimicrobium cellulans IFO 15516, Cellulosimicrobium cellulans JCM 6201, and Morganella morganii IFO 3848. Of these microorganisms, IFO 13495, IFO 15012, IFO 15014, IFO 15015, IFO 15013, IFO 15516, and IFO 3848 have already been known. These microorganisms are easily available from National Institute of Technology and Evaluation, Biological Resource Center (NBRC), (Kazusa Kamatari 2-5-8, Kisarazu, Chiba, Japan (postal code: 292-0818)). JCM 2471 and JCM 6201 have also already been known. These microorganisms are easily available from Riken Bioresource Center, Japan Collection of Microorganisms (JCM) (Hirosawa 2-1, Wako, Saitama, Japan (postal code: 351-0198)). Other microorganisms were newly separated from the soil and identified by the present inventors. The thus identified microorganisms were then independently deposited with National Institute of Advanced Industrial Science and Technology (AIST Tsukuba, Central 6, Higashi 1-1-1, Tsukuba, Ibaraki, Japan (postal code: 305-8566)) under accession numbers as described above. The mycological properties of the aforementioned Stenotrophomonas sp. KNK235 (hereinafter simply referred to as KNK235 at times), Pseudomonas sp. KNK058 (hereinafter simply referred to as KNK058 at times), Pseudomonas sp. KNK254 (hereinafter simply referred to as KNK254 at times), and Microbacterium sp. KNK011 (hereinafter simply referred to as KNK011 at times) are shown in Table 1.

TABLE 1

| | KNK235 | KNK254 | KNK058 | KNK011 |
|---|---|---|---|---|
| | Bacillus | Bacillus | Bacillus | Bacillus |
| Form of cells | 0.8 × 2.0 to 3.0 μm) | 0.7 to 0.8 × 2.0 to 2.5 μm) | 0.8 × 1.5 to 2.0 μm) | 0.7 to 0.8 × 1.0 to 1.2 μm) |
| Gram staining | − | − | − | + |
| Spore formation | − | − | − | − |
| Mobility | + | + | + | − |
| Form of colony | Round shape, smooth | Round shape, smooth | Round shape, smooth | Round shape, smooth |
| | entire fringe, | entire fringe, | entire fringe, | entire fringe, |
| | small degree of | small degree of | small degree of | small degree of |
| | convex, lustrous, | convex, lustrous, | convex, lustrous, | convex, lustrous, |
| | yellow | yellow | yellow | yellow |
| Culture temperature | +(37° C.) | −(37° C.) | +(37° C.) | +(37° C.) |
| | −(45° C.) | −(45° C.) | −(45° C.) | −(45° C.) |
| Catalase | + | + | + | + |
| Oxidase | − | + | − | − |
| OF test (glucose) | − | − | − | − |
| Nitrate reduction | − | − | − | − |
| Pyrazinamidase | | | | + |
| Pyrrolidonyl allyl amidase | | | | − |
| β-glucuronidase | | | | − |
| β-galactosidase | + | + | | + |
| α-glucosidase | | | | + |
| N-acetyl-β-glucosaminidase | | | | − |
| Esculin (β-glucosidase) | | + | | + |
| Arginine dihydrase | − | − | − | |
| Cytochrome oxidase | − | + | − | |
| Urease | − | − | − | − |
| Gelatin hydrolysis | + | + | + | + |
| Generation of indole | − | − | − | |
| Fermentability | | | | |
| Glucose | | + | + | + |
| Ribose | | | | − |
| Xylose | | | | + |
| Mannitol | | − | | + |
| Maltose | + | + | − | + |
| D-mannose | + | + | + | |
| L-arabinose | − | − | + | |
| D-mannitol | − | − | + | |
| N-acetyl-D-glucosamine | + | − | − | |
| Potassium gluconate | − | − | + | |
| n-capric acid | − | − | + | |
| Adipic acid | − | − | − | |
| Malic acid | | + | + | |
| Sodium citrate | + | + | + | |
| Phenyl acetate | − | − | − | |
| Lactose | | | | − |
| Saccharose | | | | + |
| Glycogen | | | | − |

Streptomyces sp. KNK269 (hereinafter simply referred to as KNK269 at times) has been identified by the following publicly known method. An approximately 500-bp region on the 5′-terminal side of a 16S ribosomal RNA gene (16Sr DNA) of the above strain was amplified by PCR, and the nucleotide sequence thereof was then determined. Thereafter, homologous search was carried out by a method of producing a molecular cladogram, using MicroSeq Bacterial 500 library v. 0023 database (Applied Biosystems, Calif., U.S.A.).

When the aforementioned aldehyde oxidase is produced by microorganisms belonging to the genus Streptomyces, the aldehyde oxidase preferably has the following physicochemical properties (1) to (3):
(1) optimum pH: 6 to 9;
(2) heat stability: the aldehyde oxidase retains activity of 90% or more after it has been treated at pH 7.2 at 60° C. for 20 minutes; and
(3) molecular weight: the aldehyde oxidase has a molecular weight of approximately 110,000 in gel filtration analysis, and has three subunit proteins with molecular weights of approximately 25,000, approximately 35,000, and approximately 80,000 in SDS-polyacrylamide gel electrophoresis analysis.

Among microorganisms belonging to the genus Streptomyces, Streptomyces sp. KNK269 (FERM BP-08556) is preferable.

When the aforementioned aldehyde oxidase is produced by microorganisms belonging to the genus Pseudomonas, the aldehyde oxidase preferably has the following physicochemical properties (1) to (3):
(1) molecular weight: approximately 150,000 in gel filtration analysis;
(2) optimum reaction temperature: 60° C. to 70° C.; and
(3) optimum reaction pH: 5 to 7.

Among microorganisms belonging to the genus Pseudomonas, Pseudomonas sp. KNK058 (FERM BP-08555) is preferable.

When the aforementioned aldehyde oxidase is produced by microorganisms belonging to the genus Microbacterium inside and outside of the cells thereof, the aldehyde oxidase preferably has the following physicochemical properties: molecular weight: a single protein has a molecular weight of approximately 110,000 in SDS-polyacrylamide gel electrophoresis analysis.

Among microorganisms belonging to the genus Microbacterium, Microbacterium sp. KNK011 (FERM BP-08554) is preferable.

When the aforementioned aldehyde oxidase is produced by microorganisms belonging to the genus Cellulosimicrobium inside and outside of the cells thereof, the aldehyde oxidase preferably has the following physicochemical properties: molecular weight: a single protein has a molecular weight of approximately 90,000 to 100,000 in SDS-polyacrylamide gel electrophoresis analysis.

Among microorganisms belonging to the genus Cellulosimicrobium, Cellulosimicrobium cellulans IFO 15516 is preferable.

In the present invention, a medium used for culturing microorganisms that can produce oxidoreductase that can convert glyoxal into glyoxylic acid is not particularly limited, as long as the above microorganisms can proliferate therein. An example of such a medium used herein may be a common liquid medium, which comprises: carbon sources including sugars such as glucose or sucrose, alcohols such as ethanol, glycerol, ethylene glycol, or propylene glycol, aldehydes such as glyoxal, fatty acids such as oleic acid or stearic acid and the esters thereof, and oils such as rapeseed oil or soybean oil; nitrogen sources such as ammonium sulfate, sodium nitrate, peptone, casamino acid, yeast extract, meat extract, or corn steep liquor; inorganic salts such as magnesium sulfate, sodium chloride, calcium carbonate, dipotassium hydrogen phosphate, or potassium dihydrogen phosphate; and other components such as malt extract or meat extract.

The process for producing glycolic acid of the present invention is characterized in that it comprises allowing any one selected from the group consisting of the aforementioned oxidoreductase, a culture broth containing a microorganism that can produce the above described oxidoreductase, cells separated from the culture broth, a processed product thereof, and a supernatant of a culture broth in a case where the above oxidase is produced even outside of a microorganism, to react with glyoxal, so as to convert it into glyoxylic acid and accumulate it.

Herein, the term “processed product of microorganism ” is used to mean a freeze-dried cells, an acetone-dried cells, a disrupted product of such cells, a crude enzyme solution, or the like. The term “crude enzyme solution” includes a solution obtained by disrupting or lysing cells by physical disruption methods using glass beads or the like or by biochemical methods using enzymes or the like, a cell-free extract obtained by removing solids from the above solution by centrifugation or the like, and so on. Moreover, the term “crude enzyme solution” further includes an enzyme obtained by partially purifying the aforementioned cell-free extract, using dialysis, ammonium sulfate precipitation, or chromatography, singly or in combination. Furthermore, such a processed product of microorganism may be immobilized by known means, before use. Such immobilization can be carried out by methods publicly known to persons skilled in the art (for example, crosslinking method, physical absorption method, encapsulation method, etc.).

Reaction conditions are different depending on an enzyme used, a microorganism used, or a processed product thereof. Optimum reaction conditions are as follows. The temperature is between 10° C. and 80° C., and preferably between 20° C. and 40° C. from the viewpoint of heat stability. The pH is between pH 4 and 12, and preferably between pH 6 and 10 from the viewpoint of pH stability. The reaction is preferably carried out under conditions consisting of shaking and agitation.

When the reaction is catalyzed by oxidase, hydrogen peroxide is generated in the reaction system. Hydrogen peroxide may inactivate enzymes or may decompose glyoxylic acid into formic acid. Such hydrogen peroxide generated as a result of the reaction can be decomposed and removed by addition of catalase, so as to prevent inactivation of enzymes or decomposition of glyoxylic acid.

As an enzyme of the present invention, an enzyme exhibiting only low activity to glyoxylic acid is desirable. In particular, the activity of the enzyme of the present invention to glyoxylic acid is preferably one-tenth of or less than, more preferably one-twentieth of or less than, and further preferably one-hundredth of or less than the activity thereof to glyoxal. If the activity of oxidase to glyoxylic acid exceeds one-tenth of the activity thereof to glyoxal, glyoxal is oxidized, and the generated glyoxylic acid is further oxidized. As a result, it is likely that glyoxylic acid is not accumulated in the reaction system or that the amount of glyoxylic acid accumulated is decreased. Thus, the oxidase of the present invention is characterized in that it does not only convert glyoxal into glyoxylic acid, but also its activity to glyoxylic acid is low. Glyoxal oxidase generated by wood-rotting fungi, which reportedly exhibit activity to glyoxal, exhibits high activity also to glyoxylic acid. Table 2 shows the activities of the enzymes of the present invention and enzyme generated by wood-rotting fungi to glyoxal and to glyoxylic acid. When compared with that activity to glyoxal, the enzymes of the present invention exhibit extremely low activity to glyoxylic acid.

TABLE 2

| | Relative activity (%) | | | | | |
|---|---|---|---|---|---|---|
| | | KNK | KNK | KNK | KNK | IFO |
| Substrate | P. chrysosporium | 235 | 269 | 058 | 011 | 15516 |
| Glyoxal | 100 | 100 | 100 | 100 | 100 | 100 |
| Glyoxylic | 60 | 5 | 0.1 | 0.1 | 0.5 | 1 |
| acid | | | | | | |

Moreover, the present invention relates to DNA encoding the aforementioned aldehyde oxidase, which can be used for efficient production of the aforementioned aldehyde oxidase using genetic recombination. Specifically, the present invention relates to DNA encoding the subunit of aldehyde oxidase, which has the nucleotide sequence represented by SEQ ID NOS: 4, 5, or 6. Further, DNA hybridizing with a DNA having a nucleotide sequences that is complementary to the DNA having the above nucleotide sequence under stringent conditions is also included in the DNA of the present invention, as long as a protein having a protein encoded by the above DNA as a subunit has activity of converting glyoxal into glyoxylic acid.

Furthermore, the present invention also relates to DNA encoding aldehyde oxidase, which has the nucleotide sequence represented by SEQ ID NO: 9, 10, 13, or 14. Further, DNA hybridizing with a DNA consisting of a nucleotide sequence that is complementary to the DNA consisting of the above nucleotide sequence under stringent conditions is also included in the DNA of the present invention, as long as a protein encoded by the above DNA has activity of converting glyoxal into glyoxylic acid.

Still further, DNA encoding a protein comprising any amino acid sequence resulting from deletion, substitution, or addition of one or several amino acids in the amino acid sequence represented by SEQ ID NO: 1, 2, or 3, is also included in the DNA of the present invention, as long as a protein having the protein encoded by the above DNA as a subunit has activity of converting glyoxal into glyoxylic acid.

Still further, DNA encoding a protein comprising an amino acid sequence resulting from deletion, substitution, or addition of one or several amino acids in the amino acid sequence represented by SEQ ID NO: 7, 8, 11, or 12, is also included in the DNA of the present invention, as long as the protein encoded by the above DNA has activity of converting glyoxal into glyoxylic acid.

The present invention also relates to aldehyde oxidase having a protein encoded by DNA having the nucleotide sequence represented by SEQ ID NO: 4, 5, or 6 as a subunit, and to aldehyde oxidase encoded by DNA having the nucleotide sequence represented by SEQ ID NO: 9, 10, 13, or 14. Moreover, aldehyde oxidase that has, as a subunit, a protein encoded by DNA hybridizing with any one DNA consisting of a nucleotide sequence that is complementary to the DNA consisting of the nucleotide sequence represented by SEQ ID NOS: 4, 5, or 6, under stringent conditions, and aldehyde oxidase encoded by DNA hybridizing with any one DNA consisting of a nucleotide sequence that is complementary to the DNA consisting of the nucleotide sequence represented by SEQ ID NOS: 9, 10, 13, or 14, under stringent conditions, are also included in the oxidase of the present invention, as long as they have activity of converting glyoxal into glyoxylic acid.

Hybridization can be carried out by operations that have publicly been known to persons skilled in the art. Specifically, hybridization can be carried out by Southern hybridization, using, as probe DNA, DNA obtained by end-labeling double-stranded DNA having the nucleotide sequence represented by SEQ ID NO: 4, 5, 6, 9, 10, 13, or 14, with ³²P according to the nick translation method, for example (refer to Molecular Cloning, 3^{rd} edition, 2001, Cold Spring Harbor Laboratory Press, Cold Spring Harbor, N.Y., U.S.A., Vol. 1, Chapter 6, 50-55). Chromosomal DNA, genomic DNA, or plasmid DNA, which is produced from any given organism or microorganism, artificially produced vector DNA, or a DNA fragment obtained by digesting the above DNAs with appropriate restriction enzymes, is isolated by agarose gel electrophoresis. Thereafter, the isolated DNA is immobilized onto a nitrocellulose filter, and DNA binding to the above described probe DNA is then detected by autoradiography, thereby detecting a gene of aldehyde oxidase that can be used in the present invention. Stringent conditions applied to the hybridization mean that the DNA immobilized onto the nitrocellulose filter is allowed to hybridize with the labeled probe DNA at 68° C. in a buffer consisting of 6×SSC, 5× Denhart's reagent, 0.5% SDS, 1 μg/m poly (A), and 100 μg/ml salmon sperm DNA, and then that the resultant is rinsed with a buffer consisting of 2×SSC and 0.5% SDS, followed by washing twice with a buffer consisting of 2×SSC and 0.1% SDS at 30° C. for 30 minutes. More stringent conditions include the case where the above washing is carried out 4 times with a buffer consisting of 1×SSC and 0.5% SDS at 65° C. for 30 minutes. 1×SSC is an aqueous solution containing 0.15 M sodium chloride and 0.015 M sodium citrate. 1× Denhart's reagent contains 0.02% Ficoll 400 (manufactured by Sigma-Aldrich Corporation), 0.02% polyvinylpyrrolidone, and 0.02% bovine serum albumin (manufactured by Sigma-Aldrich Corporation; Fraction V).

Furthermore, the present invention also relates to aldehyde oxidase having a protein consisting of the amino acid sequence represented by SEQ ID NO: 1, 2, or 3 as a subunit, and aldehyde oxidase having the amino acid sequence represented by SEQ ID NO: 7, 8, 11, or 12. Still further, a protein having an amino acid sequence resulting from deletion, substitution, or addition of one or several amino acids in any one of the amino acid sequences of the subunits γ, β, and α of aldehyde oxidase represented by SEQ ID NOS: 1, 2, and 3, is also included in the oxidase of the present invention, as long as such a protein having the above protein as a subunit has activity of converting glyoxal into glyoxylic acid. Still further, a protein having an amino acid sequence resulting from deletion, substitution, or addition of one or several amino acids in any one of the amino acid sequences represented by SEQ ID NOS: 7, 8, 11, and 12, is also included in the oxidase of the present invention, as long as it has activity of converting glyoxal into glyoxylic acid.

As a method of deletion, substitution, or addition of a specific amino acid(s), a conventionally known method is used. Examples of such a method may include: PCR using synthetic DNA primers having nucleotide sequences comprising deletion of codons of specific amino acids, substitution with codons of other amino acids, or addition of codons of other amino acids; and a method of ligating an enzyme gene produced by known methods such as a chemical DNA synthesis method to a gene expression vector, and allowing it to express in a host such as Escherichia coli by genetic recombination. These methods can easily be carried out by persons skilled in the art.

In the case of an enzyme secreted outside of the cells of microorganism, a secretory signal sequence is generally encoded in a portion corresponding to several tens of amino acids from the initiation codon of the gene. It has been known that such a secretory signal sequence is cleaved so as to become a mature enzyme during a step in which an enzyme protein synthesized in the cells is secreted to outside of the cells. In the case of several enzymes described in the present invention, the active enzymes are secreted also to outside of the cells. In the production of these enzymes by genetic recombination, an enzyme protein can be produced inside of the cells of a microorganism used as a host, using a gene from which a secretory signal sequence has artificially been removed. The present invention also provides enzyme genes (SEQ ID NOS: 10 and 14), from which secretory signal sequences have been removed and which can be used for the above described purpose. On the other hand, for the purpose of producing an enzyme of the cells of a host microorganism in the expression of the enzyme by genetic recombination, genes comprising secretory signal sequences (SEQ ID NOS: 9 and 13) can also be used. Further, a chimeric gene, the original secretory signal sequence of which has been substituted with another secretory signal sequence that is suitable for a host microorganism, can be constructed and used for the above described purpose by persons skilled in the art according to known methods.

The gene of an enzyme usable in the present invention can be obtained by the following method. That is to say, an enzyme protein is purified from a microorganism that produces an enzyme converting glyoxal into glyoxylic acid, or from a culture broth thereof. Thereafter, using a peptide obtained by digesting the enzyme protein with protease, partial amino acid sequences are determined. Subsequently, using primers synthesized based on these partial amino acid sequences, PCR is carried out with genomic DNA as a template according to publicly known methods. By such PCR, a part of the enzyme gene is amplified, and the nucleotide sequence of the inside of the gene can be determined. Also, inverse PCR is carried out using DNA primers synthesized from an N-terminal amino acid sequence and an amino acid sequence around the C-terminus, so as to determine a signal sequence, an N-terminal amino acid sequence, a C-terminal amino acid sequence, etc. (Cell Science 1990, vol. 6, No. 5, 370-376). The gene of aldehyde oxidase, the nucleotide sequence of which has been determined, can easily be obtained by PCR. Moreover, it is also possible to obtain such an aldehyde oxidase gene from the chromosomal DNA or genomic DNA of a microorganism by known methods using a partial sequence of the gene.

An enzyme used in the present invention may be either a natural enzyme or an enzyme obtained by recombinant technology. As such recombinant technology, a method of inserting an enzyme gene into a plasmid vector or a phage vector, and transforming a host including bacteria such as Escherichia coli, microorganisms such as yeast or fungi, or cells of such animals or plants, with the above vector, is effective, for example.

The present invention will be described more in detail in the following examples. However, these examples are not intended to limit the present invention.

### EXAMPLE 1

5 ml of liquid medium (EG-NB medium (pH 7)) with the composition consisting of 10 g of ethylene glycol, 1 g of yeast extract, 8 g of NUTRIENT BROTH (manufactured by Difco), 3 g of potassium dihydrogen phosphate, and 7 g of dipotassium hydrogen phosphate (all of which were amounts contained in 1 liter), was poured into a large test tube, and it was then sterilized by autoclaving at 121° C. for 20 minutes. Using an inoculating loop, each of the microorganisms shown in Table 3 was aseptically inoculated into this medium. It was then cultured at 28° C. for 2 days, so as to obtain a preculture broth. Subsequently, 1 ml of the obtained preculture broth was inoculated into 100 ml of the sterilized EG-NB medium placed in a 500-ml Sakaguchiflask, and it was then cultured at 28° C. for 3 days. The cells were collected from 100 ml of the obtained culture broth by centrifugation. The cells were washed with a 100 mM Tris-HCl buffer (pH 8.0) and were then suspended in 5 ml of the same above buffer (pH 8.0). The cell suspension was disrupted with Mini Beat-Beater (manufactured by BIOSPEC) and followed by centrifugation, so as to obtain a supernatant (a cell-free extract). 0.1 ml of a 500 mM glyoxal aqueous solution and 0.1 ml of a solution containing 50,000 U/ml catalase were added to 0.8 ml of the obtained cell-free extract, and the obtained mixture was subjected to a shaking reaction in a test tube at 28° C. for 4 hours. The obtained reaction solution was analyzed by high performance liquid chromatography. The amount of glyoxylic acid produced is summarized in Table 3.

TABLE 3

| | Amount of glyoxylic |
|---|---|
| Strain | acid produced (mM) |
| Stenotrophomonas sp. KNK235 | 2 |
| Streptomyces sp. KNK269 | 5 |
| Pseudomonas sp. KNK254 | 10 |
| Pseudomonas sp. KNK 058 | 12 |
| Microbacterium sp. KNK011 | 24 |
| Achromobacter sp. IFO 13495 | 2 |
| Cellulomonas sp. JCM 2471 | 32 |
| Cellulomonas turbata IFO 15012 | 13 |
| Cellulomonas turbata IFO 15014 | 15 |
| Cellulomonas turbata IFO 15015 | 20 |
| Cellulosimicrobium cellulans IFO 15013 | 14 |
| Cellulosimicrobium cellulans IFO 15516 | 23 |
| Cellulosimicrobium cellulans JCM 6201 | 21 |
| Morganella morganii IFO 3848 | 7 |

### EXAMPLE 2

0.05 ml of 0.1 M phosphate buffer (pH 7) containing 1.34 mM 4-AA, 2.19 mM TOOS, and 6 U/ml POD was added to 0.1 ml of the cell-free extract of each of the microorganisms shown in Table 3, which had been prepared in Example 1, in a test tube. Thereafter, 0.05 ml of 100 mM glyoxal aqueous solution or water was added thereto, and the mixture was shaken at 28° C. for 2 minutes. Thereafter, a change in the color of the reaction solution was observed. The results are shown in Table 4. In all the reactions of using any one of the above-described microorganisms, the reaction solution obtained when a glyoxal aqueous solution had been added exhibited a strong violet color. When water was added instead of such a glyoxal aqueous solution, the reaction solution did not change color. It was found that hydrogen peroxide is generated during the oxidization reaction of glyoxal. From this fact, it was found that an enzyme catalyzing the oxidization reaction of glyoxal is oxidase.

TABLE 4

Coloration of
reaction solution

| | Glyoxal | Glyoxal |
|---|---|---|
| Strain | added | not added |
| Stenotrophomonas sp. KNK 235 | + | − |
| Streptomyces sp. KNK 269 | + | − |
| Pseudomonas sp. KNK 254 | + | − |
| Pseudomonas sp. KNK 058 | + | − |
| Microbacterium sp. KNK011 | + | − |
| Achromobacter sp. IFO 13495 | + | − |
| Cellulomonas sp. JCM 2471 | + | − |
| Cellulomonas turbata IFO 15012 | + | − |
| Cellulomonas turbata IFO 15014 | + | − |
| Cellulomonas turbata IFO 15015 | + | − |
| Cellulosimicrobium cellulans IFO 15013 | + | − |
| Cellulosimicrobium cellulans IFO 15516 | + | − |
| Cellulosimicrobium cellulans JCM 6201 | + | − |
| Morganella morganii IFO 3848 | + | − |

### EXAMPLE 3

A culture broth of each of Pseudomonas sp. KNK254, Microbacterium sp. KNK 011, Cellulomonas turbata IFO 15015, and Cellulomonas sp. JCM2471 was prepared by the same method as described in Example 1. Thereafter, the cells were collected from 100 ml of the obtained culture broth by centrifugation, and were then washed with a 0.1 mM phosphate buffer (pH 7). Thereafter, the cells were suspended in 5 ml of the same above buffer. Thereafter, 0.05 ml of 500 mM glyoxal aqueous solution was added to 0.45 ml of the present cell suspension placed in a test tube, and the obtained mixture was reacted by shaking for 4 hours. After completion of the reaction, the obtained supernatant was analyzed by HPLC, and the amount of glyoxylic acid produced was calculated. As a result, it was found that 20 mM glyoxylic acid was produced from Pseudomonas sp. KNK 254, 14 mM glyoxylic acid was produced from Microbacterium sp. KNK011, 30 mM glyoxylic acid was produced from Cellulomonas turbata IFO 15015, and 33 mM glyoxylic acid was produced from Cellulomonas sp. JCM 2471.

### EXAMPLE 4

Aldehyde oxidase having activity of converting glyoxal into glyoxylic acid was purified from Streptomyces sp. KNK 269 by the following method.

50 ml of a medium (pH 7) with the composition consisting of 10 g of ethylene glycol, 3 g of yeast extract, 8 g of Nutrient Broth, 3 g of dipotassium hydrogen phosphate, and 7 g of dipotassium hydrogen phosphate (all of which were amounts contained in 1 liter), was poured into a 500-ml Sakaguchi flask, and it was then sterilized by autoclaving. Using an inoculating loop, Streptomyces sp. KNK 269 was inoculated into this medium. It was then subjected to shake culture at 28° C. for 3 days, so as to obtain a preculture broth. Subsequently, 6 L of the medium with the above composition was placed in a 10-L mini jar, and it was then sterilized by autoclaving. Thereafter, 50 ml of the obtained preculture broth was inoculated into the above medium, and it was then cultured at 28° C. at aeration of 0.5 vvm at agitation of 300 rpm for 2 days. This mini jar culture was repeated, so as to obtain 95 L of a culture broth. Subsequently, the cells were collected from 95 L of the obtained culture broth by centrifugation, and they were then suspended in 3 L of a 0.05 M phosphate buffer (pH 7).

The obtained cell suspension was disrupted with Dyno Mill (manufactured by Dyno-Mill), and cell residues were then removed by centrifugation, so as to obtain 2.5 L of a cell-free extract. While stirring with a stirrer under cooling on ice, a predetermined amount of ammonium sulfate was added to 2.5 L of the obtained cell-free extract. Thereafter, proteins precipitated with 30% to 55% saturation of ammonium sulfate were collected by centrifugation.

The obtained proteins were dissolved in a 0.05 M phosphate buffer (pH 7), and dialysis was carried out with the same buffer. The resultant solution was charged to a DEAE-TOYOPEARL 650 M column (manufactured by Tosoh Corporation) (130 ml) that had previously been equilibrated with the same buffer, and fractions that passed by the column were eliminated. Thereafter, the remaining protein was eluted with a 0.05 M phosphate buffer (pH 7) containing 0.5 M sodium chloride, so as to collect active fractions. Thereafter, ammonium sulfate was added to the obtained enzyme solution such that the concentration of ammonium sulfate became 0.6 M. The mixed solution was charged to a Phenyl-TOYOPEARL 650 M column (manufactured by Tosoh Corporation) (300 ml) that had previously been equilibrated with a 0.05 M phosphate buffer containing 0.6 M ammonium sulfate. Thereafter, the enzyme was eluted with a linear concentration gradient of ammonium sulfate from 0.6 to 0.1 M, so as to collect active fractions. The obtained enzyme, solution was dialyzed with a 0.05 M phosphate buffer (pH 7). The resultant solution was charged to a DEAE-TOYOPEARL 650 M column (130 ml) that had previously been equilibrated with the same above buffer. Thereafter, the enzyme was eluted with a linear concentration gradient of sodium chloride from 0 to 0.25 M, so as to collect active fractions. Thereafter, ammonium sulfate was added thereto until it became 60% saturated. Precipitated proteins were collected by centrifugation and were then dissolved in a 0.05 M phosphate buffer (pH 7), followed by dialysis with the same buffer. Subsequently, the enzyme solution obtained after the dialysis was charged to a Benzamidine Sepharose column (manufactured by Amersham Pharmacia Biotech) (10 ml) that had previously been equilibrated with a 0.05 M phosphate buffer (pH 7). Thereafter, the enzyme was eluted with a linear concentration gradient of sodium chloride from 0 to 0.1 M, so as to collect active fractions. This enzyme solution was concentrated by ultrafiltration. The concentrate was then charged to a Superdex 200HR 16/60 column (manufactured by Amersham Pharmacia Biotech) (120 ml) that had previously been equilibrated with a 0.05 M phosphate buffer (pH 7) containing 0.15 M sodium chloride. Thereafter, the solution was eluted with the same above buffer. An elution peak with the same activity as that of protein absorption at 280 nm was obtained from a fraction corresponding to a molecular weight of 170,000. When this active fraction was subjected to native polyacrylamide gel electrophoresis, it formed a single band.

On the other hand, when the present enzyme was subjected to SDS-polyacrylamide gel electrophoresis, it formed three protein bands corresponding to molecular weights of approximately 25,000, 35,000, and 80,000. From these results, it was found that the present enzyme has a structure consisting of subunits with molecular weights of approximately 25,000, 35,000, and 80,000.

### EXAMPLE 5

Aldehyde oxidase having activity of converting glyoxal into glyoxylic acid was purified from Microbacterium sp. KNK 011 by the following method.

50 ml of medium (pH 7) with the composition consisting of 5 g of yeast extract, 2 g of ammonium nitrate, 2 g of dipotassium hydrogen phosphate, 1 g of sodium dihydrogen phosphate dihydrate, 0.2 g of magnesium sulfate heptahydrate, and 0.1 g of calcium chloride dihydrate (all of which were amounts contained in 1 liter), was poured into a 500-ml flask, and it was then sterilized by autoclaving. Using an inoculating loop, Microbacterium sp. KNK 011 was inoculated into this medium. It was then subjected to shake culture at 28° C. for 3 days, so as to obtain a preculture broth. Subsequently, 3 L of the medium with the above composition was placed in a 5-L mini jar, and it was then sterilized by autoclaving. Thereafter, 30 ml of the obtained preculture broth was inoculated into the above medium, and it was then cultured at 28° C. at aeration of 0.5 vvm at agitation of 400 rpm for 28 hours. This mini jar culture was repeated, so as to obtain 69 L of a culture broth. Subsequently, the cells were collected from 69 L of the obtained culture broth by centrifugation, and they were then suspended in 0.05 M phosphate buffer (pH 7). The obtained cells suspension was disrupted with Dyno Mill (manufactured by Dyno-Mill), and cell residues were then removed by centrifugation, so as to obtain 2 L of a cell-free extract. While stirring with a stirrer under cooling on ice, a predetermined amount of ammonium sulfate was added to 2 L of the obtained cell-free extract. Thereafter, proteins precipitated with 20% to 40% saturation of ammonium sulfate were collected by centrifugation.

The obtained proteins were dissolved in a 0.05 M phosphate buffer (pH 7), and dialysis was carried out with the same buffer. The resultant solution was charged to a DEAE-TOYOPEARL 650 M column (300 ml) that had previously been equilibrated with the same buffer. Thereafter, the enzyme was eluted with a linear concentration gradient of sodium chloride from 0 to 0.6 M, so as to collect active fractions. Thereafter, a predetermined amount of ammonium sulfate was added to the obtained active fractions such that the concentration of ammonium sulfate became 0.7 M. The mixed solution was charged to a Phenyl-TOYOPEARL 650 M column (160 ml) that had previously been equilibrated with a 0.05 M phosphate buffer (pH 7) containing 0.7 M ammonium sulfate. Thereafter, the enzyme was eluted with a linear concentration gradient of ammonium sulfate from 0.7 to 0 M, so as to collect active fractions. The obtained enzyme solution was dialyzed with a 0.05 M phosphate buffer (pH 7). Thereafter, the enzyme solution obtained after the dialysis was charged to a Resource Q column (manufactured by Amersham Pharmacia Biotech) (6 ml) that had previously been equilibrated with a 0.05 M phosphate buffer. Thereafter, the solution was eluted with linear concentration gradient of sodium chloride from 0.15 to 0.45 M, so as to collect active fractions. The obtained enzyme solution was concentrated by ultrafiltration. Thereafter, ammonium sulfate was added thereto such that the concentration thereof became 0.3 M. The obtained mixed solution was charged to a Resource Phe column (manufactured by Amersham Pharmacia Biotech) (6 ml) that had previously been equilibrated with a 0.05 M phosphate buffer (pH 7) containing 0.3 M ammonium sulfate. Thereafter, the enzyme was eluted with a linear concentration gradient of ammonium sulfate from 0.3 to 0 M, so as to collect active fractions.

The obtained enzyme solution was subjected to SDS-polyacrylamide gel electrophoresis. As a result, it formed a single protein band corresponding to a molecular weight of approximately 110,000.

### EXAMPLE 6

Aldehyde oxidase having activity of converting glyoxal into glyoxylic acid was purified from Pseudomonas sp. KNK058 by the following method.

5 ml of medium (pH 7) with the composition consisting of 10 g of ethylene glycol, 8 g of nutrient broth, 7 g of dipotassium hydrogen phosphate, and 3 g of potassium dihydrogen phosphate (all of which were amounts contained in 1 liter), was sterilized by autoclaving in a large test tube. Using an inoculating loop, Pseudomonas sp. KNK058 was inoculated into this medium, and it was cultured at 28° C. for 2 days. Subsequently, the obtained culture broth was inoculated into 500 ml of the above-sterilized medium placed in a 2-L shake flask, and thus, it was subjected to shake culture at 28° C. for 18 hours, so as to obtain a preculture broth. Subsequently, the obtained preculture broth was inoculated into 60 L of the above-sterilized medium placed in a jar fermenter, and it was then cultured at 28° C. at aeration of 1 vvm at agitation of 200 rpm for 40 hours. Thereafter, cells were collected from 60 L of the obtained culture broth by centrifugation, and they were then suspended in a 0.02 M phosphate buffer (pH 7). The obtained cells suspension was disrupted with an Inconator 201M ultrasonic disintegration device (manufactured by Kubota Corporation) for 60 minutes, and cell residues were then removed by centrifugation, so as to obtain a cell-free extract. While the obtained cell-free extract was stirred with a stirrer under cooling, a predetermined amount of ammonium sulfate was added thereto. Thereafter, proteins precipitated with 20% to 60% saturation of ammonium sulfate were collected by centrifugation.

The obtained proteins were dissolved in a 0.02 M phosphate buffer (pH 7), and dialysis was carried out with the same buffer. Thereafter, 1.5 L of DEAE-Sephacel resin was added to the enzyme solution, and the mixture was stirred at 4° C. for 1 hour. Thereafter, an unadsorbed protein solution was removed by filtration, and an enzyme protein adsorbed onto the resin was eluted with 1 M sodium chloride.

The obtained enzyme solution was dialyzed with a 0.02 M phosphate buffer (pH 7), and it was then charged to a HiPrep 16/10-Q-XL column (manufactured by Amersham Pharmacia Bioscience) (16 ml) that had previously been equilibrated with a 0.02 M phosphate buffer (pH 7), and elution was carried out with a linear concentration gradient of sodium chloride from 0 to 1 M, so as to collect active fractions. A predetermined amount of ammonium sulfate was added to the active fractions such that the concentration thereof became 1.2 M. The mixed solution was charged to a Phenyl Superose HR 10/10 column (manufactured by Amersham Pharmacia Bioscience) (10 ml) that had previously been equilibrated with a 0.02 M phosphate buffer containing 1.2 M ammonium sulfate. Thereafter, the enzyme was eluted with a linear concentration gradient of ammonium sulfate from 1.2 to 0 M, so as to collect active fractions. Thereafter, the obtained enzyme solution was dialyzed with a 0.02 M phosphate buffer. The resultant solution was charged to a MonoQ HR 10/10 column (manufactured by Amersham Pharmacia Bioscience) (10 ml) that had previously been equilibrated with the same above buffer. Thereafter, the solution was eluted with linear concentration gradient of sodium chloride, so as to collect active fractions. Also, the obtained enzyme solution was charged to a HiPrep Sephacryl S-200 16/60 column (manufactured by Amersham Pharmacia Bioscience) (60 ml) that had previously been equilibrated with a 0.02 M phosphate buffer containing 0.02 M sodium chloride, and the solution was eluted with the same above buffer. Active fractions were collected, and they were then dialyzed with a 0.005 M phosphate buffer (pH 7). The resultant solution was charged to a Hydroxyapatite column (manufactured by Seikagaku Corporation) (10 ml) that had previously been equilibrated with the same above buffer, and the enzyme was eluted with a linear concentration gradient of a phosphate buffer from 0.005 to 0.5 M. Active fractions were collected, and they were then dialyzed with a 0.005 mM phosphate buffer. The resultant solution was charged to a Bio-Scale CHT5-I column (manufactured by Bio-Rad) (6.4 ml) that had previously been equilibrated with the same above buffer, and the enzyme was eluted with a linear concentration gradient of a phosphate buffer from 0.005 to 0.5 M, so as to collect active fractions. When the active fractions were subjected to native polyacrylamide gel electrophoresis, it formed a single band.

### EXAMPLE 7

Aldehyde oxidase having activity of converting glyoxal into glyoxylic acid was purified from the culture supernatant of Cellulosimicrobium cellulans IFO 15516 by the following method.

60 ml of medium (pH 7) with the composition consisting of 10 g of yeast extract, 2 g of ammonium sulfate, 1 g of dipotassium hydrogen phosphate, 1 g of sodium dihydrogen phosphate, 0.2 g of magnesium sulfate heptahydrate, and 0.1 g of calcium chloride dihydrate (all of which were amounts contained in 1 liter), was poured into a 500-ml Sakaguchi flask, and it was then sterilized by autoclaving. Thereafter, using an inoculating loop, Cellulosimicrobium cellulans NBRC 15516 was inoculated into this medium. It was then subjected to shake culture at 28° C. for 2 days, so as to obtain apreculture broth. Subsequently, 3 L of the medium with the above composition was placed in a 5-L mini jar, and it was then treated by autoclaving. Thereafter, 60 ml of the obtained preculture broth was inoculated into the above medium, and it was then cultured at 28° C. at aeration of 0.5 vvm at agitation of 400 rpm for 27 hours. The same culture was repeated, so as to obtain 45 L of a culture broth. The obtained culture broth was adjusted to be pH 7, and it was then centrifuged, so as to obtain 45 L of a culture supernatant. The obtained culture supernatant was concentrated to 2.4 L using agitation-type Ultra Holder UHP150 (manufactured by Advantec Toyo) . Thereafter, while the concentrate was stirred under cooling on ice, a predetermined amount of ammonium sulfate was added thereto. Thereafter, proteins precipitated with 0% to 60% saturation of ammonium sulfate were collected by centrifugation. The obtained proteins were dissolved in a 20 mM potassium phosphate buffer (pH 7), and dialysis was carried out with a sufficient amount of the same buffer. The resultant solution was charged to a DEAE-TOYOPEARL 650 M column (300 ml) that had previously been equilibrated with the same above buffer. Thereafter, the enzyme was eluted with a linear concentration gradient of sodium chloride from 0 to 0.5 M, so as to collect active fractions. Thereafter, a predetermined amount of ammonium sulfate was added to the obtained active fractions to a final concentration of 1 M. The mixed solution was charged to a Phenyl-TOYOPEARL 650 M column (60 ml) that had previously been equilibrated with a 20 mM potassium phosphate buffer (pH 7) containing 1 M ammonium sulfate. Thereafter, the enzyme was eluted with a linear concentration gradient of ammonium sulfate from 1 to 0.5 M, so as to collect active fractions. The obtained enzyme solution was dialyzed with a 20 mM potassium phosphate buffer (pH 7). Thereafter, the resultant solution was charged to a Resource Q column (manufactured by Amersham Pharmacia Biotech) (6 ml) that had previously been equilibrated with the same above buffer. Thereafter, the solution was washed with the same above buffer containing 0.35 M sodium chloride, and the enzyme was then eluted with a linear concentration gradient of sodium chloride from 0.35 to 0.5 M, so as to collect active fractions. The obtained enzyme solution was concentrated by ultrafiltration. Thereafter, the concentrate was charged to a Superdex 200HR column (manufactured by Amersham Pharmacia Biotech) (24 ml) that had previously been equilibrated with a 20 mM potassium phosphate buffer (pH 7) containing 0.15 M sodium chloride. Thereafter, the solution was eluted with the same above buffer. The obtained active fractions were subjected to SDS-polyacrylamide gel electrophoresis. As a result, it formed a single band with a molecular weight between 90,000 and 100,000.

### EXAMPLE 8

The cell-free extract of each of KNK235 and KNK058 obtained by the method described in Example 1 was charged to a Resource Q column (6 ml) that had previously been equilibrated with a 0.05 M phosphate buffer (pH 7), and the enzyme was eluted with a linear concentration gradient of sodium chloride from 0 to 0.5 M, so as to collect active fractions. Roughly purified enzyme solutions of these KNK235 and KNK058 and purified enzymes obtained from Streptomyces sp. KNK269, Microbacterium sp. KNK011, and Cellulosimicrobium cellulans IFO 15516, obtained in Examples 4, 5, and 7, respectively, were measured in terms of their oxidase activity to glyoxal and to glyoxylic acid. Measurement of the enzyme activity was carried out by the following method. That is, 1.0 ml of a reaction solution comprising 10 mM glyoxal or glyoxylic acid, 0.67 mM 4-AA, 1.09 mM TOOS, 2 U/ml POD, and a roughly purified enzyme from KNK235 or KNK058, or a purified enzyme obtained from NKN269, KNK011, or IFO 15516 in Example 4, 5, or 7, was reacted at 30° C. in a 100 mM phosphate buffer (pH 7). Thereafter, an increase in the absorbance at a wavelength of 555 nm was measured. A comparison made among the activities of the above enzymes to glyoxal and glyoxylic acid is shown in Table 5.

TABLE 5

| | Activity to glyoxal/activity |
|---|---|
| | to glyoxylic acid |
| KNK235-derived enzyme | 19 |
| KNK058-derived enzyme | 1,100 |
| KNK269-derived enzyme | 1,710 |
| KNK011-derived enzyme | 183 |
| IFO15516-derived enzyme | 100 |

The activity of each enzyme to glyoxylic acid was lower than that to glyoxal.

### EXAMPLE 9

The physicochemical properties of the enzyme obtained from Streptomyces sp. KNK269 in Example 4 were examined. Measurement of the enzyme activity was basically carried out by the method described in Example 8.

(Optimum pH)

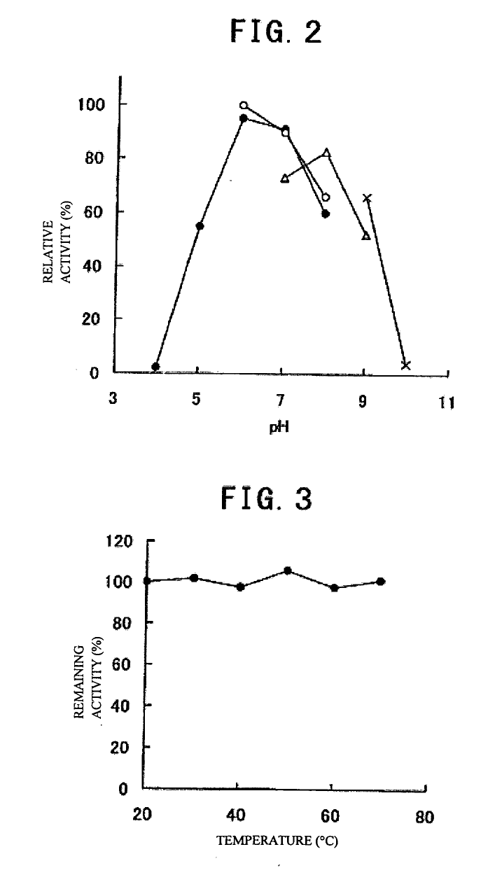

Activity was measured using glyoxal as a substrate within a range between pH 5 and 9. The results are shown in FIG. 2. It was found that the optimum pH is between 6 and 9.

(Heat Stability)

The present enzyme was treated in a 0.05 M phosphate buffer (pH 7.2) at each temperature from 30° C. to 70° C. for 20 minutes. Thereafter, the remaining activity of the present enzyme was measured using glyoxal as a substrate. The results are shown in FIG. 3. It was found that 90% or more of the activity remained after the treatment at 70° C.

### Example 10

The physicochemical properties of the enzyme obtained from Pseudomonas sp. KNK058 in Example 6 were examined. Measurement of the enzyme activity was basically carried out by the method described in Example 8.

(Molecular Weight)

The molecular weight of the present enzyme was measured using TSK-G3000SW column (manufactured by Tosoh Corporation). As a result, it was found to be approximately 150,000.

(Optimum Temperature)

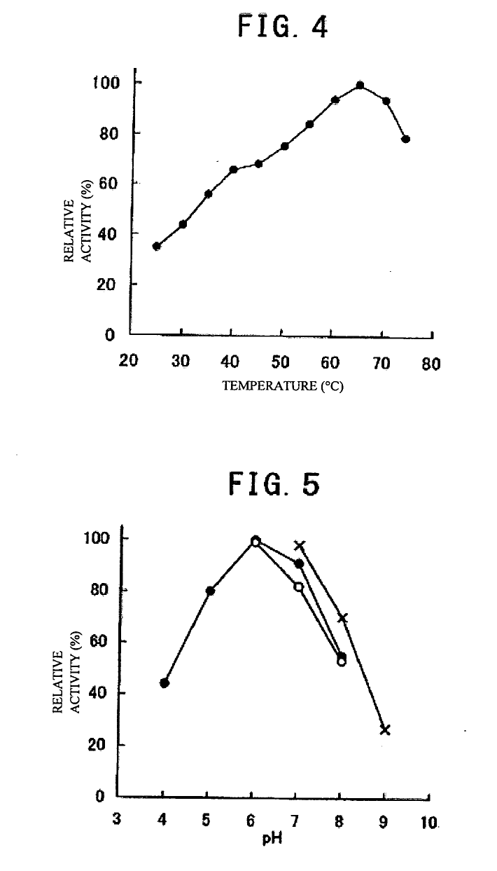

Activity was measured using glyoxal as a substrate within a temperature range between 25° C. and 75° C. The results are shown in FIG. 4. The enzyme exhibited high activity in a temperature range between 60° C. and 70° C.

(Optimum pH)

Using a McIlvine buffer, a phosphate buffer, or a Tricine buffer as a buffer, activity was measured with glyoxal as a substrate within a pH range between pH 4 and 9. The results are shown in FIG. 5. The enzyme exhibited high activity in a pH range between pH 5 and 7.

### EXAMPLE 11

Each of the enzymes derived from Streptomyces sp. KNK269, Microbacterium sp. KNK011, Pseudomonas sp. KNK058, and Cellulosimicrobium cellulans IFO 15516, which were obtained in Examples 4, 5, 6, and 7, respectively, was reacted using glyoxal as a substrate. That is, 1 ml of 100 mM Tris-HCl buffer comprising 0.2 U/ml enzyme derived from each of the above strains, 50 mM glyoxal, and 5,000 U/ml catalase, were placed in a test tube. It was then subjected to a shake reaction at 30° C. for 3 hours. After completion of the reaction, the reaction solution was analyzed by high performance liquid chromatography. As a result, it was found that 7.6 mM glyoxylic acid was generated from the enzyme derived from KNK269, 25 mM glyoxylic acid was generated from the enzyme derived from KNK011, 5.7 mM glyoxylic acid was generated from the enzyme derived from KNK058, and 26 mM glyoxylic acid was generated from the enzyme derived from IFO 15516.

### EXAMPLE 12

The activity of aldehyde oxidase contained in the cultured cells of Cellulomonas turbata IFO 15012, Cellulomonas turbata IF015014, Cellulosimicrobium cellulans IFO 15013, Cellulosimicrobium cellulans IFO 15516, Cellulosimicrobium cellulans JCM 6201, and Microbacterium sp. KNK011, was measured by the following method, respectively.

5 ml of medium (pH 7) with the composition consisting of 2 g of ammonium nitrate, 1 g of dipotassium hydrogen phosphate, 1.3 g of sodium dihydrogen phosphate, 5 g of yeast extract, 0.2 g of magnesium sulfate heptahydrate, and 0.1 g of calcium chloride (all of which were amounts contained in 1 liter), was poured into a large test tube, and it was then sterilized by autoclaving. The aforementioned strain was inoculated into this medium using an inoculating loop, and preculture was then carried out at 28° C. for 2 days. Subsequently, 1 ml of the obtained preculture broth was inoculated into 60 ml of the same above medium placed in a 500-ml Sakaguchi flask, and it was then cultured at 28° C. for 2 days. Thereafter, the cells were collected from the obtained culture broth by centrifugation, and they were then washed with 100 mM potassium phosphate buffer (pH 7) twice, and then suspended in 5.0 ml of the same above buffer. 0.45 ml of a 133 mM glyoxal aqueous solution and 0.05 ml of a 50,000 U/ml catalase solution were added to 1.0 ml of the present cell suspension, and the obtained mixture was subjected to a shake reaction in a test tube at 28° C. for 4hours. Thereafter, the obtained reaction solution was analyzed by high performance liquid chromatography. As a result, it was found that glyoxylic acid was generated from glyoxal in all types of the strains.

Moreover, 0.1 ml of the cell suspension was added to 0.1 ml of a 100 mM potassium phosphate buffer comprising 50 mM glyoxal, 1.34 mM 4-AA, 2.18 mM TOOS, and 4 U/ml peroxidase, and the mixture was then shaken at 28° C. for 2 hours. Thereafter, oxidase activity was evaluated by visual observation of the coloration of the reaction solution. The same above reaction solution without glyoxal was simultaneously examined as a control test. As a result, in all types of the strains, only when glyoxal was added, coloration due to oxidase activity was observed. Thus, it became clear that glyoxal was oxidized by oxidase. These results are summarized in Table 6.

TABLE 6

| | | Coloration of | |
|---|---|---|---|
| | Amount of | reaction solution | |
| | glyoxylic acid | Glyoxal | Glyoxal |
| Strain | produced (mM) | added | not added |
| Cellulomonas turbata IFO 15012 | 5.1 | +++++ | − |
| Cellulosimicrobium cellulans | 8.2 | ++++ | − |
| IFO 15013 | | | |
| Cellulomonas turbata IFO 15014 | 4.1 | +++ | − |
| Cellulosimicrobium cellulans | 22.6 | ++++ | − |
| IFO 15516 | | | |
| Cellulosimicrobium cellulans | 20.1 | ++++ | − |
| JCM 6201 | | | |
| Microbacterium sp. KNK011 | 4.0 | +++ | − |

### EXAMPLE 13

Using the supernatants of the culture broths of Microbacterium sp. KNK011 and Cellulosimicrobium cellulans IFO 15516 prepared in Example 12, the secretion of aldehyde oxidase to outside of the cells thereof was confirmed. The culture broth was centrifuged, and the culture supernatant, from which cells had been removed, was concentrated by ultrafiltration using Amicon Centriplus YM-10 (manufactured by MILLIPORE). The buffer was exchanged with a 100 mM potassium phosphate buffer (pH 7), so as to prepare a 12-times concentrate. Using 0.1 ml of the thus obtained culture supernatant concentrate, oxidase activity was evaluated by coloration using glyoxal as a substrate in the same manner as in Example 12. As a result, as shown in Table 7, the culture supernatant also exhibited enzyme activity that was equivalent to that of the cell suspension.

TABLE 7

| | Coloration of reaction | |
|---|---|---|
| | solution | |
| | | Concentrated |
| | Cell | culture |
| Strain | suspension | supernatant |
| Cellulosimicrobium cellulans IFO 15013 | ++++ | ++++ |
| Microbacterium sp. KNK011 | +++ | +++ |

### EXAMPLE 14

The amino acid sequence of aldehyde oxidase purified from Streptomyces sp. KNK269 in Example 4 was determined by the following method. Reverse phase HPLC was applied to separate three types of subunits that constitute the enzyme. As a column, YMC-Pack PROTEIN-RP column (250×4.6 mm) (manufactured by YMC) was used. As a mobile phase, 0.1% trifluoroacetic acid was used. The subunits were separated by a method of charging 300 μg of the purified enzyme to the column and eluting it with a linear concentration gradient of acetonitrile from 0% to 56% (flow rate: 1 ml/min.; 110 minutes) . A unit with a molecular weight of 25,000 was eluted at a retention time of approximately 85 minutes (hereinafter referred to as subunit γ). Another unit with a molecular weight of 35,000 was eluted at a retention time of approximately 90 minutes (hereinafter referred to as subunit β). Another unit with a molecular weight of 80,000 was eluted at a retention time of approximately 92 minutes (hereinafter referred to as subunit α). Using one-tenth of each of the separated subunits, the N-terminal amino acid sequence thereof was determined by the Edman degradation method using Protein Sequencing System 490 Procise (manufactured by Applied Biosystems). Subsequently, the internal amino acid sequence of each subunit protein was determined. The residual amount of each subunit protein as a whole was denatured with 9M urea. The buffer was exchanged with a 0.3 M Tris-HCl buffer (pH 9.0), and it was then digested with lysyl endopeptidase at 30° C. for 19 hours. The degradation products were purified by the reverse phase HPLC method using an YMC-Pack PROTEIN-RP column. 0.1% trifluoroacetic acid was used as a mobile phase, and peptides were eluted with a linear concentration gradient of acetonitrile from 10% to 48%. Each peak eluted was separated, and the amino acid sequence of the inside of the subunit was determined by the same method. Of the obtained amino acid sequences, representative examples are represented by SEQ ID NOS: 15 to 20.

### EXAMPLE 15

Based on the obtained partial amino acid sequences, mixed DNA primers (SEQ ID NOS: 21 to 26) were synthesized. Using the genomic DNA of Streptomyces sp. KNK269 as a template, PCR was carried out with the above mixed DNA primers in a GC buffer (manufactured by Takara Shuzo Co., Ltd.), with TAKARA LA Taq DNA polymerase (manufactured by Takara Shuzo Co., Ltd.). The obtained amplified DNA was subjected to agarose gel electrophoresis, and DNA that formed a band was extracted using QIAquick Gel Extraction kit (manufactured by QIAGEN). The obtained DNA was directly sequenced. Otherwise, it was TA cloned into pT7Blue-2 (manufactured by Novagen), and DNA sequencing was then carried out using a plasmid, so as to determine a nucleotide sequence. PCR was carried out using the primers represented by SEQ ID NOS: 21 to 26 in the combination with (1) and (2), (3) and (4), and (5) and (6), so as to obtain amplified DNA sequences. The thus obtained sequences were converted into amino acids, and these amino acids were checked against the amino acid sequences determined in Example 14. They were then aligned. As a result, it was found that genes encoding the three types of subunits were adjacent to one another on the genome in the order of γ, α, and β from the upstream, or a portion thereof overlapped. The nucleotide sequences of the mixed primer portions were determined by the same above method using peripheral nucleotide sequences as primers. Thus, the total nucleotide sequences of genomic regions encoding all the genes of the subunits γ, α, and β were determined, except for the sequence around the N-terminus of the subunit γ and the sequence around the C-terminus of the subunit α. The amino acid sequences obtained from the obtained nucleotide sequences completely matched with the partial amino acid sequences of the subunits obtained in Example 14. The determined amino acid sequences of the subunits γ, α, and β are represented by SEQ ID NOS: 1 to 3. The nucleotide sequences thereof are represented by SEQ ID NOS: 4 to 6. SEQ ID NOS: 1 and 4 indicate an amino acid sequence from the N-terminus of the purified protein of the subunit γ and a nucleotide sequence from Glu12 to the termination codon thereof, respectively. SEQ ID NOS: 2 and 5 indicate the entire amino acid sequence of the subunit β and the entire nucleotide sequence from the initiation codon to the termination codon thereof, respectively. SEQ ID NOS: 3 and 6 indicate an amino acid sequence from Met 1 to Thr 693 of the subunit α and a nucleotide sequence from the initiation codon to Arg 685 thereof, respectively. The N-terminal amino acid sequence of the subunit α of the enzyme purified from KNK269 represented by SEQ ID NO: 18 started with Ala 5 of the amino acid sequence represented by SEQ ID NO: 3 that was determined from the gene arrangement.

### EXAMPLE 16

The amino acid sequence of aldehyde oxidase purified from Microbacterium sp. KNK011 in Example 5 was determined. Using 10 μg of the purified enzyme that had been desalted with an ultrafilter membrane, the N-terminal amino acid sequence thereof was determined by the same method as applied in Example 14. Subsequently, in order to determine the amino acid sequence of the inside of the protein, the amino acid sequence of the protease-digested peptide of the purified enzyme was determined. 100 μg of the purified enzyme was denatured with 9 M urea. The buffer was exchanged with a 0.2 M Tris-HCl buffer (pH 9.0), and it was then digested with lysyl endopeptidase at 30° C. for 16 hours. The thus obtained digested peptide mixture was separated by the reverse phase HPLC method using an YMC-Pack PROTEIN-RP column (250×4.6 mm) by the same method as in Example 14. Each peak eluted was separated, and the amino acid sequence was determined by the same method, so as to determine the partial amino acid sequence of the inside of the enzyme protein. Of the obtained amino acid sequences, representative examples are represented by SEQ ID NOS: 27 to 29.

### EXAMPLE 17

Based on the determined partial amino acids, mixed DNA primers (SEQ ID NOS: 30 to 33) were synthesized. The genomic DNA of Microbacterium sp. KNK011 was used as a template, and PCR was carried out by the same method as in Example 15. The amplified DNA was subjected to agarose gel electrophoresis, and DNA that formed a band was extracted using QIAquick Gel Extraction kit (manufactured by QIAGEN). The obtained DNA was directly sequenced. Otherwise, it was TA cloned into pT7Blue-2, and DNA sequencing was then carried out, so as to determine a nucleotide sequence. PCR was carried out using the primers represented by SEQ ID NOS: 30 to 33 in the combination with (1) and (2), and (3) and (4). The thus obtained nucleotide sequences were checked against the amino acid sequences. The approximately 2.8-kb nucleotide sequence of the purified enzyme was determined by the same method as in Example 15, except for the sequences around the N- and C-termini thereof. The nucleotide sequences around the N- and C-termini of the purified enzyme were determined by the inverse PCR method (refer to Cell Science, 1990, Vol. 6, No. 5, 370-376). The genomic DNA was treated with various types of restriction enzymes, and self-ligation was then carried out at a final concentration of 2.5 ng/ml. Using primers synthesized from the nucleotide sequences around the above termini, PCR was carried out. The obtained amplified DNA was ligated to pT7Blue-2, and the nucleotide sequence thereof was determined in the same manner. A sequence from upsteam of the initiation codon to downstream of the N-terminus of the purified enzyme was determined from an amplified band consisting of approximately 650 nucleotides that had been obtained by inverse PCR using the PvuI digest of the genomic DNA. On the other hand, a nucleotide sequence around the termination codon was determined from an amplified band consisting of approximately 1.9-kb nucleotides that had been obtained by inverse PCR using the ApaI digest of the genomic DNA. The thus determined full-length gene from the initiation codon to the termination codon consisted of 3,348 nucleotides, and also consisted of 1,115 residues in terms of amino acids. An amino acid sequence obtained from the above nucleotide sequence completely matched with the amino acid sequence obtained by the amino acid sequence analysis of the purified enzyme. The N-terminus of the enzyme purified from the cells was Val at position 47from the initiation codon Met 1. Since Microbacterium sp. KNK011 produced the same enzyme in the supernatant of the culture broth thereof, it was thereby found that a portion from Met 1 to Ala 46 is cleaved as a secretory signal sequence during the secretion process. The determined entire amino acid sequence of the enzyme protein and the determined entire nucleotide sequence of the enzyme gene are represented by SEQ ID NOS: 7 and 9, respectively. The entire amino acid sequence of the mature enzyme after secretion and the nucleotide sequence encoding the above enzyme are represented by SEQ ID NOS: 8 and 10, respectively.

### EXAMPLE 18

The gene of aldehyde oxidase obtained from Microbacterium sp. KNK011 that had been sequenced in Example 17 was cloned into an expression vector, and an expression experiment was then carried out. The codon of Ala 46 was substituted with an initiation codon atg. A synthesis primer to which a restriction enzyme NdeI recognition sequence had been added (SEQ ID NO: 34), and a synthesis primer of a complementary sequence wherein a restriction enzyme EcoRI recognition sequence had been added to 63 to 68 nucleotides downstream of the termination codon (SEQ ID NO: 35), were used. Using the genomic DNA of Microbacterium sp. KNK011 as a template, PCR was carried out with the above primers, so as to obtain an amplified band with approximately 3.7 kb. DNA that formed this band was subjected to agarose gel electrophoresis, and then extracted with QIAquick Gel Extraction kit (manufactured by QIAGEN), followed by digestion with NdeI and EcoRI. The digested DNA was subjected to agarose gel electrophoresis, and extraction was then carried out in the same above manner. The obtained extract was ligated to an expression vector pUCNT (Japanese Patent Laid-Open No. 2003-116552) that had been digested with NdeI and EcoRI. Thereafter, E. coli DH5α was transformed with the resultant product. The obtained transformant was cultured overnight in an LB medium containing 100 μg of ampicillin. Thereafter, the culture was transferred to a fresh medium of the same type, and then cultured. Two hours later, 1 mM IPTG was added thereto, and the culture was carried out for 5 hours. Thereafter, the cultured cells were treated with SDS, and the entire protein was then subjected to SDS-polyacrylamide gel electrophoresis. As a result, a band of enzyme protein was confirmed at a position of approximately 11 kDa.

### EXAMPLE 19

The amino acid sequence of aldehyde oxidase purified from Cellulosimicrobium cellulans IFO 15516 in Example 7 was determined. Using 10 μg of the purified enzyme that had been desalted with an ultrafilter membrane, the amino acid sequence thereof was determined using Protein Sequencing System Model 490 Procise (manufactured by Applied Biosystems) Subsequently, 100 μg of the purified enzyme was denatured with 9 M urea. The buffer was exchanged with a 0.2 M Tris-HCl buffer (pH 9.0), and it was then digested with lysyl endopeptidase at 30° C. for 16 hours. The lysate was purified by the reverse phase HPLC method using an YMC-Pack PROTEIN-RP column (manufactured by YMC). As a mobile phase, 0.1% trifluoroacetic acid was used, and peptides were eluted with a linear concentration gradient of acetonitrile from 10% to 55%. Each peak eluted was separated, and the amino acid sequence of the inside of the protein was determined in the same manner. Of the obtained amino acid sequences, representative examples are represented by SEQ ID NOS: 36 to 38.

### EXAMPLE 20

Mixed DNA primers (SEQ ID NOS: 39 to 41) synthesized based on the partial amino acids determined in Example 19, and a mixed DNA primer (SEQ ID NO: 42) of the complementary strand DNA consisting of 2521 to 2545 nucleotides of the aldehyde oxidase gene of Microbacterium sp. KNK011, were used. Using the genomic DNA of Cellulosimicrobium cellulans IFO 15516 as a template, PCR was carried out with the above primers. The amplified DNA obtained by PCR using the primers represented by SEQ ID NOS: 39 to 42 in combination with (1) and (2), and (3) and (4), was subjected to agarose gel electrophoresis, and DNA that formed a band was extracted using QIAquick Gel Extraction kit (manufactured by QIAGEN). The obtained DNA was directly sequenced. Otherwise, it was TA cloned into pT7Blue-2, and thereafter, DNA sequencing was carried out, so as to determine a nucleotide sequence. The thus obtained nucleotide sequence was checked against the amino acid sequence. The approximately 2.5-kb nucleotide sequence of the purified enzyme was determined by the same method as in Example 15, except for the sequences around the N- and C-termini thereof. The nucleotide sequences around the N- and C-termini of the purified enzyme were determined by the same inverse PCR method as in Example 17. The genomic DNA was treated with various types of restriction enzymes, and self-ligation was then carried out at a final concentration of 2.5 ng/ml. Using primers synthesized from the nucleotide sequences around the above termini, PCR was carried out. The obtained amplified DNA was ligated to pT7Blue-2, and the nucleotide sequence thereof was determined in the same manner. A sequence from upsteam of the initiation codon to downstream of the N-terminus of the purified enzyme was determined from an amplified band with approximately 1 kb that had been obtained by inverse PCR using the NaeI digest of the genomic DNA. On the other hand, a nucleotide sequence around the termination codon was determined from an approximately 1.1-kb amplified band that had been obtained by inverse PCR using the NcoI digest of the genomic DNA. The thus determined full-length gene from the initiation codon to the termination codon consisted of 3,324 nucleotides, and also consisted of 1,107 residues in terms of amino acids. An amino acid sequence obtained from the above nucleotide sequence completely matched with the amino acid sequence obtained by the amino acid sequence analysis of the purified enzyme. The N-terminus of the enzyme purified from the cells was Asp at position 39 from the initiation codon Met 1. In the case of enzyme secreted into the medium, a portion from Met 1 to Ala 38 was cleaved as a secretory signal sequence during the secretion process. The determined entire amino acid sequence of the enzyme protein and the determined entire nucleotide sequence of the enzyme gene are represented by SEQ ID NOS: 11 and 13, respectively. The entire amino acid sequence of the mature enzyme after secretion and the nucleotide sequence encoding the above enzyme are represented by SEQ ID NOS: 12 and 14, respectively.

### INDUSTRIAL APPLICABILITY

The process for production of glyoxylic acid from glyoxal using microorganisms or enzymes of the present invention enables production of glyoxylic acid under moderate conditions. It enables production of glyoxylic acid without generation of a large amount of salts, which has been problematic in the conventional chemical synthesis methods such as the nitric acid oxidation method.

Sequence Listing Free Text

SEQ ID NO: 1 Amino acid sequence of subunit γ of aldehyde oxidase
SEQ ID NO: 2 Amino acid sequence of subunit β of aldehyde oxidase
SEQ ID NO: 3 Amino acid sequence of subunit α of aldehyde oxidase
SEQ ID NO: 4 DNA sequence of subunit γ of aldehyde oxidase
SEQ ID NO: 5 DNA sequence of subunit β of aldehyde oxidase
SEQ ID NO: 6 DNA sequence of subunit α of aldehyde oxidase
SEQ ID NO: 7 Amino acid sequence of aldehyde oxidase containing signal peptide
SEQ ID NO: 8 Amino acid sequence of aldehyde oxidase
SEQ ID NO: 9 DNA sequence of aldehyde oxidase containing signal peptide
SEQ ID NO: 10 DNA sequence of aldehyde oxidase
SEQ ID NO: 11 Amino acid sequence of aldehyde oxidase containing signal peptide
SEQ ID NO: 12 Amino acid sequence of aldehyde oxidase
SEQ ID NO: 13 DNA sequence of aldehyde oxidase containing signal peptide
SEQ ID NO: 14 DNA sequence of aldehyde oxidase
SEQ ID NO: 15 N-terminal amino acid sequence of subunit γ of aldehyde oxidase
SEQ ID NO: 16 N-terminal amino acid sequence of subunit β of aldehyde oxidase
SEQ ID NO: 17 Amino acid sequence of subunit β of aldehyde oxidase (Ala 231 to Ala 266)
SEQ ID NO: 18 N-terminal amino acid sequence of subunit α of aldehyde oxidase
SEQ ID NO: 19 Amino acid sequence of subunit α of aldehyde oxidase (Leu 261 to Glu 298)
SEQ ID NO: 20 Amino acid sequence of subunit α of aldehyde oxidase (Gly 659 to Thr 693)
SEQ ID NO: 21 Mixed DNA primer (1) corresponding to N-terminal amino acid sequence of subunit γ of aldehyde oxidase derived from Streptomyces sp. KNK269
SEQ ID NO: 22 Complementary mixed DNA primer (2) corresponding to amino acid sequence (Asp 251 to Ala 258) of subunit β of aldehyde oxidase derived from Streptomyces sp. KNK269
SEQ ID NO: 23 Mixed DNA primer (3) corresponding to amino acid sequence (Asp 251 to Ala 258) of subunit β of aldehyde oxidase derived from Streptomyces sp. KNK269
SEQ ID NO: 24 Complementary mixed DNA primer (4) corresponding to amino acid sequence (Leu 274 to Glu 281) of subunit α of aldehyde oxidase derived from Streptomyces sp. KNK269
SEQ ID NO: 25 Mixed DNA primer (5) corresponding to amino acid sequence (Leu 274 to Glu 281) of subunit α of aldehyde oxidase derived from Streptomyces sp. KNK269
SEQ ID NO: 26 Complementary mixed DNA primer (6) corresponding to amino acid sequence (Leu 686 to Glu 693) of subunit α of aldehyde oxidase derived from Streptomyces sp. KNK269
SEQ ID NO: 27 N-terminal amino acid sequence of aldehyde oxidase
SEQ ID NO: 28 Internal amino acid sequence of aldehyde oxidase
SEQ ID NO: 29 Internal amino acid sequence of aldehyde oxidase
SEQ ID NO: 30 Mixed DNA primer (1) corresponding to N-terminal amino acid sequence of aldehyde oxidase derived from Microbacterium sp. KNK011
SEQ ID NO: 31 Complementary mixed DNA primer (2) corresponding to amino acid sequence (Asp 513 to Phe 521) of aldehyde oxidase derived from Microbacterium sp. KNK011
SEQ ID NO: 32 Mixed DNA primer (3) corresponding to amino acid sequence (Asp 513 to Phe 521) of aldehyde oxidase derived from Microbacterium sp. KNK011
SEQ ID NO: 33 Complementary mixed DNA primer (4) corresponding to amino acid sequence (Phe 959 to Thr 969) of aldehyde oxidase derived from Microbacterium sp. KNK011
SEQ ID NO: 34 DNA primer containing NdeI restriction site, which is used for cloning of aldehyde oxidase derived from Microbacterium sp. KNK011
SEQ ID NO: 35 DNA primer containing EcoRI restriction site, which is used for cloning of aldehyde oxidase derived from Microbacterium sp. KNK011
SEQ ID NO: 36 N-terminal amino acid sequence of aldehyde oxidase
SEQ ID NO: 37 Internal amino acid sequence of aldehyde oxidase
SEQ ID NO: 38 Internal amino acid sequence of aldehyde oxidase
SEQ ID NO: 39 Mixed DNA primer (1) corresponding to N-terminal amino acid sequence of aldehyde oxidase derived from Cellulosimicrobium cellulans IFO 15516
SEQ ID NO: 40 Complementary mixed DNA primer (2) corresponding to amino acid sequence (Ile 350 to Val. 358) of aldehyde oxidase derived from Cellulosimicrobium cellulans IFO 15516
SEQ ID NO: 41 Mixed DNA primer (3) corresponding to amino acid sequence (Thr 176 to Thr 183) of aldehyde oxidase derived from Cellulosimicrobium cellulans IFO 15516
SEQ ID NO: 42 Complementary mixed DNA primer (4) corresponding to DNA sequence (2521 to 2545) of aldehyde oxidase derived from Microbacterium sp. KNK011

1. A process for production of glyoxylic acid, which comprises allowing oxidoreductase that can convert glyoxal into glyoxylic acid, or at least one or a mixture consisting of two or more selected from the group consisting of a culture broth, a supernatant of the culture broth, cells, and processed products of microorganism that can produce the oxidoreductase, to act on glyoxal, so as to convert glyoxal into glyoxylic acid.

2. The process for production of glyoxylic acid according to claim 1, wherein the oxidoreductase is oxidase.

3. The process for production of glyoxylic acid according to claim 1 or 2, wherein the oxidoreductase is obtained from at least one microorganism selected from the group consisting of the genus Stenotrophomonas, Streptomyces, Pseudomonas, Microbacterium, Achromobacter, Cellulomonas, Cellulosimicrobium, and Morganella.

4. The process for production of glyoxylic acid according to claim 3, wherein the microorganism is Stenotrophomonas sp. KNK235 (FERM P-19002), Streptomyces sp. KNK269 (FERM BP-08556), Pseudomonas sp. KNK058 (FERM BP-08555), Pseudomonas sp. KNK254 (FERM P-19003), Microbacterium sp. KNK011 (FERM BP-08554), Achromobacter sp. IFO 13495, Cellulomonas sp. JCM 2471, Cellulomonas turbata IFO 15012, Cellulomonas turbata IFO 15014, Cellulomonas turbata IFO 15015, Cellulosimicrobium cellulans IFO 15013, Cellulosimicrobium cellulans IFO 15516, Cellulosimicrobium cellulans JCM 6201, or Morganella morganii IFO 3848.

5. The process for production of glyoxylic acid according to claim 1, wherein catalase is allowed to coexist during the reaction.

6. An aldehyde oxidase derived from a microorganism that acts on glyoxal to generate glyoxylic acid.

7. The aldehyde oxidase according to claim 6, whose activity to glyoxylic acid is one-tenth or less of its activity to glyoxal.

8. The aldehyde oxidase according to claim 6 or 7, wherein the aldehyde oxidase is produced by at least one microorganism selected from the group consisting of the genus Stenotrophomonas, Streptomyces, Pseudomonas, Microbacterium, Achromobacter, Cellulomonas, Cellulosimicrobium, and Morganella.

9. The aldehyde oxidase according to claim 8, wherein the microorganism is Stenotrophomonas sp. KNK235 (FERM P-19002), Streptomyces sp. KNK269 (FERM BP-08556), Pseudomonas sp. KNK058 (FERMBP-08555), Pseudomonas sp. KNK254 (FERM P-19003), Microbacterium sp. KNK011 (FERM BP-08554), Achromobacter sp. IFO 13495, Cellulomonas sp. JCM 2471, Cellulomonas turbata IFO 15012, Cellulomonas turbata IFO 15014, Cellulomonas turbata IFO 15015, Cellulosimicrobium cellulans IFO 15013, Cellulosimicrobium cellulans IFO 15516, Cellulosimicrobium cellulans JCM 6201, or Morganella morganii IFO 3848.

10. The aldehyde oxidase according to claim 8, which is produced by a microorganism belonging to the genus Streptomyces and which has the following physicochemical properties (1) to (3):
(1) optimum pH: 6 to 9;
(2) heat stability: the aldehyde oxidase retains activity of 90% or more after it has been treated at pH 7.2 at 60° C. for 20 minutes; and
(3) molecular weight: the aldehyde oxidase has a molecular weight of approximately 110,000 in gel filtration analysis, and has three subunit proteins with molecular weights of approximately 25,000, approximately 35,000, and approximately 80,000 in SDS-polyacrylamide gel electrophoresis analysis.

11. The aldehyde oxidase according to claim 8, which is produced by a microorganism belonging to the genus Pseudomonas and which has the following physicochemical properties (1) to (3):
(1) molecular weight: approximately 150,000 in gel filtration analysis;
(2) optimum reaction temperature: 60° C. to 70° C.; and
(3) optimum reaction pH: 5 to 7.

12. The aldehyde oxidase according to claim 8, which is generated by a microorganism belonging to the genus Microbacterium inside and outside of the cells thereof and which has the following physicochemical properties: molecular weight: a single protein has a molecular weight of approximately 110,000 in SDS-polyacrylamide gel electrophoresis analysis.

13. The aldehyde oxidase according to claim 8, which is generated by a microorganism belonging to the genus Cellulosimicrobium inside and outside of the cells thereof, and which has the following physicochemical properties: molecular weight: a single protein has a molecular weight of approximately 90,000 to 100,000 in SDS-polyacrylamide gel electrophoresis analysis.

14. The aldehyde oxidase according to claim 10, wherein the microorganism belonging to the genus Streptomyces is Streptomyces sp. KNK269 (FERM BP-08556).

15. The aldehyde oxidase according to claim 11, wherein the microorganism belonging to the genus Pseudomonas is Pseudomonas sp. KNK058 (FERM BP-08555).

16. The aldehyde oxidase according to claim 12, wherein the microorganism belonging to the genus Microbacterium is Microbacterium sp. KNK011 (FERM BP-08554).

17. The aldehyde oxidase according to claim 13, wherein the microorganism belonging to the genus Cellulosimicrobium is Cellulosimicrobium cellulans IFO 15516.

18. The aldehyde oxidase according to claim 6, which has a protein described in the following (a) or (b) as a subunit:
(a) a protein having an amino acid sequence represented by SEQ ID NO: 1, 2, or 3; or
(b) a protein comprising an amino acid sequence result from deletion, substitution, or addition of one or several amino acids in the amino acid sequence (a).

19. The aldehyde oxidase according to claim 6, which has a protein encoded by the DNA described in the following (a) or (b) as a subunit:
(a) DNA having a nucleotide sequence represented by SEQ ID NO: 4, 5, or 6; or
(b) DNA which hybridizes with any one DNA consisting of a nucleotide sequence that is complementary to the DNA consisting of the nucleotide sequence (a) under stringent conditions.

20. The aldehyde oxidase according to claim 6, which has the amino acid sequence described in the following (a) or (b):
(a) an amino acid sequence represented by SEQ ID NO: 7, 8, 11, or 12; or
(b) an amino acid sequence resulting from deletion, substitution, or addition of one or several amino acids in the amino acid sequence (a).

21. The aldehyde oxidase according to claim 6, which is encoded by the DNA described in the following (a) or (b):
(a) DNA having a nucleotide sequence represented by SEQ ID NO: 9, 10, 13, or 14; or
(b) DNA which hybridizes with DNA consisting of a nucleotide sequence that is complementary to the DNA consisting of the nucleotide sequence (a) under stringent conditions.

22. DNA encoding a subunit of the aldehyde oxidase according to claim 6, which comprises the DNA described in the following (a) or (b):
(a) DNA having a nucleotide sequence represented by SEQ ID NO: 4, 5, or 6; or
(b) DNA which hybridizes with any one DNA consisting of a nucleotide sequence that is complementary to the DNA consisting of the nucleotide sequence (a) under stringent conditions.

23. DNA encoding the aldehyde oxidase according to claim 6, which comprises the DNA described in the following (a) or (b):
(a) DNA having a nucleotide sequence represented by SEQ ID NO: 9, 10, 13, or 14; or
(b) DNA which hybridizes with any one DNA consisting of a nucleotide sequence that is complementary to the DNA having the nucleotide sequence (a) under stringent conditions.

24. DNA encoding a subunit of the aldehyde oxidase according to claim 6, which comprises an amino acid sequence resulting from deletion, substitution, or addition of one or several amino acids in the amino acid sequence represented by SEQ ID NO: 1, 2, or 3.

25. DNA encoding the aldehyde oxidase according to claim 6, which comprises an amino acid sequence resulting from deletion, substitution, or addition of one or several amino acids in the amino acid sequence represented by SEQ ID NO: 7, 8, 11, or 12.
